(19) United States
(12) Patent Application Publication
(10) Pub. No.: US 2025/0265710 A1
(43) Pub. Date: Aug. 21, 2025

(54) NON-INVASIVE LASER SPECKLE IMAGING OF EXTRA-EMBRYONIC BLOOD VESSELS

(71) Applicant: California Institute of Technology, Pasadena, CA (US)

(72) Inventors: Zhenyu Dong, Pasadena, CA (US); Simon Mahler, Pasadena, CA (US); Carol Readhead, Pasadena, CA (US); Changhuei Yang, South Pasadena, CA (US)

(21) Appl. No.: 19/056,622

(22) Filed: Feb. 18, 2025

Related U.S. Application Data
(60) Provisional application No. 63/725,421, filed on Nov. 26, 2024.
(60) Provisional application No. 63/725,450, filed on Nov. 26, 2024.
(60) Provisional application No. 63/554,060, filed on Feb. 15, 2024.

(51) Int. Cl. G06T 7/00; G01N 33/08

(52) U.S. Cl. CPC G06T 7/0012; G01N 33/08; G06T 2207/20081; G06T 2207/20084; G06T 2207/30044; G06T 2207/30104

(57) ABSTRACT

Laser speckle contrast imaging techniques that apply a temporal sliding window to a sequence of speckle frames of an avian egg to determine overlapping sets of speckle frames used to reconstruct a sequence of extraembryonic blood vessel images (e.g., movie) showing blood flow dynamics, and where input to a machine learning model based on the overlapping sets of speckle frames can be used to predict the developmental stage of the avian egg, and where the overlapping sets of speckle frames can be used in drug screening.

### CROSS-REFERENCES TO RELATED APPLICATION

This application claims priority to and benefit of U.S. Provisional Patent Application No. 63/554,060, filed on Feb. 15, 2024 and titled “Non-Invasive Laser Speckle Imaging of Extra-Embryonic Blood Vessels in Intact Few-Days-Old Avian Eggs,” U.S. Provisional Patent Application No. 63/725,421, filed on Nov. 26, 2024 and titled “Non-Invasive Laser Speckle Imaging of Extra-Embryonic Blood Vessels in Intact Few-Days-Old Avian Eggs,” and U.S. Provisional Patent Application No. 63/725,450, filed on Nov. 26, 2024 and titled “Automated Non-Invasive Laser Speckle Imaging of the Chick Heart Rate and Extraembryonic Blood Vessels and their Response to Drugs Injection;” all of which are hereby incorporated by reference in their entireties and for all purposes.

### FIELD

Certain aspects generally relate to techniques for laser speckle contrast imaging and more specifically to laser speckle imaging techniques that may be used to noninvasively image extraembryonic blood vessel dynamics.

### BACKGROUND

Chickens are a convenient, nutritious, and inexpensive source of food. Moreover, chicken embryos, mainly due to their accessibility, have served as fundamental research models in developmental biology as well as for the pharmaceutical industry, vaccine development, and agriculture.

### SUMMARY

Techniques disclosed herein may be practiced with a processor-implemented method, a system comprising one or more processors and one or more processor-readable media, and/or one or more non-transitory processor-readable media.

Embodiments pertain to laser speckle contrast imaging methods. In some cases, laser speckle contrast imaging methods include causing, using one or more laser sources, light to be emitted into a sample, obtaining, using one or more light detectors, a sequence of speckle frames indicative of light scattered by one or more dynamic elements within the sample, applying a temporal sliding window of a plurality of speckle frames to the sequence of speckle frames to determine overlapping sets of speckle frames, and reconstructing a sequence of dynamic element images from corresponding overlapping sets of speckle frames. In some cases, the sample includes an avian egg. In one case, a laser speckle contrast imaging method extracts one or more blood vessel features from the sequence of dynamic element images, provides the one or more blood vessel features as an input into a trained machine learning model, and predicts the developmental stage of the avian embryo based on an output of the trained machine learning model.

Embodiments pertain to methods of drug screening. In some cases, these method include (a) injecting a first drug into a set of first eggs, (b) injecting a second drug into a set of second eggs, (c) obtaining a sequence of blood vessel images based on temporal speckle contrast of each egg of the first eggs and each of the second eggs, (d) extracting a plurality of features from each of the blood vessel images for each of the first eggs and second eggs, and (e) determining a time-varying blood vessel feature metric of each of the features based on the blood vessel images.

Embodiments pertain to methods of training a machine learning model to predict a developmental stage of an avian embryo. In some cases, the methods obtain training data, the training data comprising, representations of blood vessel metrics, wherein for each training sample, a portion of the training data spans an exposure time and comprises one or more blood vessel images based on temporal speckle contrast, and wherein each training sample includes a corresponding ground truth developmental stage for the avian embryo. The methods also provide the training data to a machine learning model, wherein the machine learning model takes, as input, the representations of blood vessel metrics and generates, as an output, the prediction of developmental stage. In addition, the methods update the machine learning model based on differences between a ground truth developmental stage and the predicted developmental stage to generate a trained machine learning model configured to predict developmental stage.

Embodiments pertain to laser speckle contrast imaging systems for non-invasively imaging a plurality of avian eggs. The laser speckle contrast imaging systems include one or more laser sources, a plurality of light detectors, an integrated incubator, and one or more processors. The one or more processors are configured to (a) cause, using the one or more laser sources, light to be emitted into one of the avian eggs, (b) obtain, using the one or more light detectors, a sequence of speckle frames indicative of light scattered by one or more dynamic elements within the one of the avian eggs, (c) applying a temporal sliding window of a plurality of speckle frames to the sequence of speckle frames to determine overlapping sets of speckle frames, and (d) reconstructing a sequence of blood vessel images of the one of the avian eggs from corresponding overlapping sets of speckle frames.

These and other features are described in more detail below with reference to the associated drawings.

### BRIEF DESCRIPTION OF THE DRAWINGS

FIG. 1A is an illustration of an example of the development of vascularized extraembryonic membranes in a 6-7 day chick embryo, according to various embodiments.

FIG. 1B is a diagram of an example of an extraembryonic vascular network of a 4-day chick embryo, according to embodiments.

FIG. 2 is a simplified block diagram of components of an LSCI system, according to various embodiments.

FIG. 3 is a simplified block diagram of components of a dual-channel LSCI system, according to embodiments.

FIG. 4 is a simplified block diagram of components of an LSCI system with an integrated incubator, according to embodiments.

FIG. 5 is a schematic representation of speckle dynamics of living tissues, according to an embodiment.

FIG. 6 is a flowchart of an example LSCI process for generating a sequence of dynamic element images, according to embodiments.

FIG. 7 is a schematic illustration depicting examples of temporal sliding window being applied, according to implementations.

FIG. 8 is a schematic diagram of an example LSCI process for generating a sequence of blood vessel images of an avian egg (e.g., movie of extraembryonic blood vessels), according to some embodiments.

FIG. 9 is a flowchart depicting an example LSCI process for estimated a dark noise frame, according to an embodiment.

FIG. 10 is a flowchart depicting an example LSCI process for determining heart rate, according to an embodiment.

FIG. 11A depicts example images of the four Hamburger-Hamilton (HH) developmental stages: HH15-16, HH17-18, HH19-20, and HH21-22, according to embodiments.

FIG. 11B depicts example blood vessel images determined with LSCI methods with and without ADP filtering, and with a conventional wide-field microscope for comparison, according to embodiments.

FIG. 12 is a block diagram of an example system for classifying a developmental stage of an avian embryo in accordance with some embodiments.

FIG. 13 is a flowchart of an example LSCI process for extracting features from a blood vessel image, according to embodiments.

FIG. 14 is a flowchart of an example LSCI process for extracting features from a blood vessel image, according to embodiments.

FIG. 15 depicts illustrations of examples of extracted features, according to embodiments.

FIG. 16 is a flowchart of an example process for determining a developmental stage using blood vessel data in accordance with some embodiments.

FIG. 17 is a flowchart of an example process for training a machine learning model in accordance with some embodiments.

FIG. 18 is a diagram depicting results of testing different classifiers, according to embodiments.

FIG. 19A is a confusion matrix, according to embodiments.

FIG. 19B is a plot of the average accuracy of using SVC model as compared to three human tests, according to embodiments.

FIG. 20 is a plot a 2D visualization of the data point distributions in feature space consisting of the first two principal components using principal component analysis, according to embodiments.

FIG. 21 is a flowchart of an example LSCI process for drug screening, according to some embodiments.

FIG. 22 is a schematic representation of a procedure for using LSCI imaging to determine the location of avian embryos for injection, according to an embodiment.

FIG. 23A depicts images of the extraembryonic blood vessels of a control egg, according to an embodiment.

FIG. 23B depicts images of the extraembryonic blood vessels of an egg injected with Nifedipine, according to an embodiment.

FIG. 24 depicts extraembryonic blood vessel images before and after feature extraction, and a heartbeat signal, according to embodiments.

FIG. 25A includes a plot of heart rate, a plot of length of the extraembryonic blood vessels, plot of the number of branches and plot of the size of the area vaculosa (AV) of test eggs injected with three different Nifedipine concentrations, according to embodiments.

FIG. 25B includes a plot of heart rate, a plot of length of the extraembryonic blood vessels, plot of the number of branches, and plot of the size of the area vaculosa (AV) of test eggs injected two different Amlodipine concentrations, according to embodiments.

FIG. 26 includes a plot of heart rate, a plot of length of the extraembryonic blood vessels, the number of branches, and the size of the area vaculosa of five test eggs injected with different concentrations of Nifedipine according to embodiments.

FIG. 27 is a table of the smallest blood vessel diameters for five eggs determined by an LSCI system and a conventional microscope for comparison, according to embodiments.

FIG. 28 is a table of photographic images of six chicken eggs, brightfield transmission images of the six chicken eggs, and images of the six chicken eggs generated by LSCI system according to an embodiment.

FIG. 29 is a table of photographic images of six quail eggs, brightfield transmission images of the six quail eggs, and images of the six quail eggs generated by LSCI system according to an embodiment.

FIG. 30A depicts images of white eggs across three incubation days: day 3 (72 hours), day 4 (96 hours), and day 5 (120 hours).

FIG. 30B depicts images of brown eggs across three incubation days: day 3 (72 hours), day 4 (96 hours), and day 5 (120 hours).

FIG. 31A depicts three snapshots from a blood flow index movie, according to embodiments.

FIG. 31B depicts a plot of the heartbeat signal of normalized blood flow and a plot of the heartbeat frequency, according to an embodiment.

FIG. 31C depicts a plot of the heartbeat signal of normalized blood flow and a plot of the heartbeat frequency using the speckle visibility spectroscopy (SVS) method.

FIG. 31D depicts a plot of the heartbeat signal of normalized blood flow and a plot of the heartbeat frequency taken by opening up the egg and recording light microscopy images of the embryo.

FIG. 32 illustrates an example computing device, according to embodiments.

The figures and components therein may not be drawn to scale.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

### I. Introduction

Chickens (and other avians) are not only valuable for the study of embryonic development, but also as model systems for biomedical research. During a chick embryo's development, a rich network of blood vessels grows in the extraembryonic membranes that provides a means for gas and waste exchange. The chick embryo follows a specific developmental process that may be categorized into Hamburger and Hamilton stages or “HH-stages,” with each stage corresponding to distinct developmental features of the embryo. At the tenth stage (HH10 at approximately 33 h of incubation), the heart, which is just a simple tube, starts to beat and extraembryonic blood vessels begin to grow and form a network. These vessels, develop in the area vasculosa (AV) or yolk sac membrane (YSM) which covers the surface of the yolk. At a later stage (day 6-7) an extraembryonic blood vessels network is established in the chorionicallantoic (CAM) membrane. This inexpensive and accessible cardiovascular system of the chick embryo has been widely used in cardiovascular and cancer biomedical research as a model to study: heart development, angiogenesis, anti-hypertensive drugs, tumor vascularization, and anti-vasculogenic drugs. Moreover, the highly vascularized chick extraembryonic blood vessels, including those of the CAM can be used to explore the effects of drugs on a whole living and functioning organism rather than on cultured cells or organoids. The impact of drugs such as: antihypertensive agents, heart teratogens and factors that either inhibit or enhance vasculogenesis can be studied under normal or pathologenic physiological conditions.

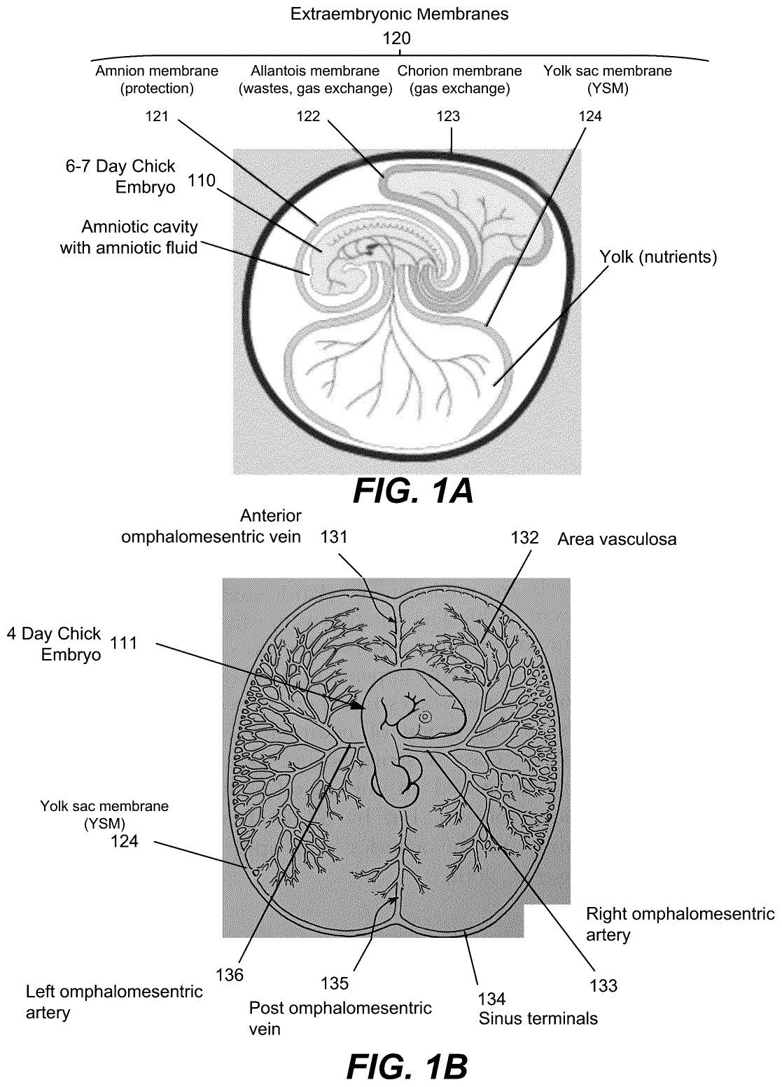

FIG. 1A depicts an illustration of an example of a 6-7 day chick embryo 110 with vascularized extraembryonic membranes 120, according to embodiments. The vascularized extraembryonic membranes 120 include an amnion membrane 121, a yolk sac membrane (YSM) 124, and a chorioallantoic membrane (CAM) (allantois membrane 122 and chorion membrane 123). FIG. 1B depicts an illustration of an example of a vascularized yolk sac membrane (YSM) 124 of a chick embryo 111 at 4 days of incubation, according to embodiments. The vascularized yolk sac membrane (YSM) 124 includes an anterior omphalomesentric vein 131, area vasculosa 132, right omphalomesentric artery 133, sinus terminals 134, post omphalomesentric vein 135, and left omphalomesentric artery 136.

Developmental and biomedical studies on chick embryos can be done in ovo or ex ovo. For example, the embryo and blood vessels can be isolated and grown outside the eggshell in vitro. For the in ovo technique a hole is cut in the eggshell, the protective membrane is removed, and substitute it with a transparent window (e.g., with transparent tape). As this technique involves breaking the eggshell and potentially removing the outer of the embryo its natural development is altered. This alteration changes the gas exchange dynamics, cardiodynamics, physical tension on the embryo, and resistance to infection, which negatively impacts the overall survival of the embryo.

In certain embodiments, laser speckle contrast imaging (LSCI) techniques (e.g., LSCI systems and methods) can non-invasively image and monitor one or more blood vessel metrics of blood vessels in extraembryonic membranes such as the area vasculosa (AV) also called the yolk sac membrane (YSM) and the CAM without disrupting the eggshell or otherwise causing harm to the embryo's natural development. Some examples of blood vessel metrics that can be monitored include heart rate, blood flow, blood volume, and blood oxygenation. The ability to observe the growth and function of extraembryonic blood vessels non-invasively and longitudinally within the intact egg is beneficial for various scientific studies, commercial applications, and conservation.

Certain LSCI techniques described herein can image the blood vessel network of an avian embryo with non-invasiveness, good resolution, and cost effectiveness. Generally speaking, LSCI techniques use the interaction between scattered laser light and dynamic elements such as blood cells flowing through blood vessels. These interactions create speckle patterns, which can be recorded and used to reconstruct images of the dynamic elements such as blood vessel images over time. Although various embodiments described herein use coherent light from one or more lasers, it is contemplated that one or more other types of coherent light sources may be used according to other implementations.

LSCI techniques described herein can overcome the opacity of the shell and may visualize the beating heart of an avian embryo and the flow of blood in the extraembryonic vessels. In certain implementations, LSCI techniques can be used evaluate blood vessel metrics, such as blood flow (e.g., blood flow index (BFI)), heart rate, blood vessel size, etc. in real-time. Although discussed mainly with regards to avian eggs, the LSCI techniques described herein can be used to visualize blood vessels and monitor blood vessel metrics in a variety of tissues including avian eggs and human tissues such as cerebral tissues.

### Classifying Avian Embryo Development Stage

Imaging blood vessels in early-stage avian embryos has a wide range of applications for developmental biology studies, and drug discovery. Conventional imaging techniques were challenged by eggshell opacity and light scattering. Certain LSCI techniques described herein provide a high-quality, comprehensive, and non-invasive visualization of blood vessels in few-days-old chicken eggs, with blood vessels as small as 100 μm in diameter (with profile full-width-at-half-maximum of 275 μm). In certain implementations, LSCI techniques can be used to non-invasively monitor blood flow, measure the embryo's heartbeat, and determine the embryo's developmental stage using machine learning with about 85% accuracy from stage HH15 to HH22. These techniques can potentially be used for non-invasive longitudinal studies of cardiovascular development and angiogenesis, as well as egg screening for the poultry industry.

Certain LSCI techniques described herein non-invasively visualize blood vessel formation in avian eggs to evaluate embryo development and non-invasively evaluate extraembryonic blood vessel metrics. For example, the LSCI techniques can be used to construct a series of extraembryonic blood vessel images over time (e.g., a movie) based on raw speckle patterns. These LSCI techniques can be used to perform longitudinal tracking of blood vessels network development. In some cases, LSCI techniques can be used to non-invasively monitor and visualize blood vessel metrics such as, e.g., heart rate, blood flow, blood volume, and/or blood oxygenation. For example, some LSCI techniques can be used to determine blood flow dynamics and estimate the corresponding heartbeat within the egg and associated heart rate. In some cases, LSCI techniques can be used to perform physical feature extraction on the blood vessel images and provide them as input into a machine learning model (e.g., a deep neural network (DNN) such as a convolutional neural network) to determine the embryonic development stage across a plurality of different developmental stages (e.g., four different Hamilton-Hamburger (HH) stages). As discussed in Section III, certain LSCI methods described herein have been shown to advantageously enhance visualizations of blood vessel formation in few-days-old (day-2 to day-6) chicken eggs (or other avians) as compared with other imaging methods.

In certain embodiments, LSCI systems including one or more laser sources that provide illumination in a first direction onto one side of the egg (also referred to herein as “side illumination”). The one or more laser sources are configured to provide illumination that allows for high photon transmission efficiency through the eggshell. For example, a laser source with a single wavelength may be used such as, for example, a 852 nm near-infrared laser (few hundreds of milliwatts power. The LSCI systems also include an image detection system (sometimes referred to herein as a “detection imaging system”) with one or more light detectors positioned to receive scattered light from the egg from a direction that is at an angle (e.g., about 90 degrees, between 45 and 135 degrees, etc.) to the side illumination. For example, the side illumination may be directed in a horizontal direction toward the center of the egg and the light detector(s) may be positioned with the imaging plane at a horizonal plane above the egg. The image detection system may have a tunable lens for focus adjustment. The image detection system may include a high-resolution camera. In one of these certain embodiments, the LSCI system is a dual-channel system that further includes components configured to provide egg candling (sometimes referred to herein as “dual-channel LSCI system”). Egg candling generally refers to brightfield imaging using a broadband light source placed beneath or behind or above the egg and the light detectors placed on the opposing side of the egg. As the egg is held against the light source, the internal components of the egg can be visualized due to the differences in the absorption of the light. As egg candling relies on absorption of the illuminating light, the imaging can be compromised by undesired external sources of light absorption such as the cracks or pigmentations of the eggshell as well as air-bubbles, especially between the inner and outer shell membranes. In addition, precise control of the illumination power is imperative to prevent overexposure and underexposure.

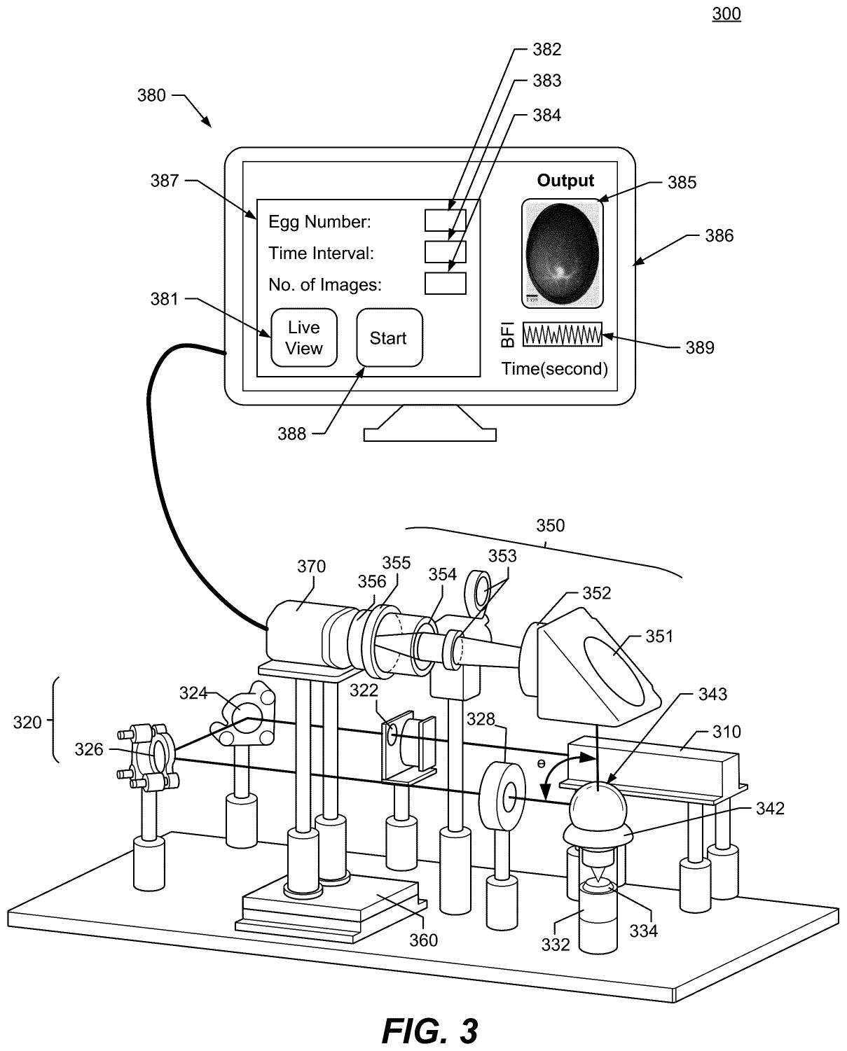

According to one implementation, a dual-channel LSCI system includes one or more broadband light sources configured to provide broadband illumination to a first side of the egg opposite a second side with the one or more light detectors. This dual-channel LSCI system integrates the broadband light source(s) to use the same or similar detection path as used to detect the scattered light from the laser source(s). This dual-channel LSCI system can be used to compare speckle images constructed from the raw speckle patterns captured under coherent illumination with bright field images captured from through transmission under broadband illumination. An example of such a dual channel system is dual-channel LSCI system 300 shown in FIG. 3. Section II discusses a comparison of speckle contrast images with bright field images captured by dual-channel LSCI system 300 in FIG. 3 with LEDs that provide broadband light for LED egg candling. The comparison shows that speckle contrast imaging is more resistance to variations in eggshell color and shade as compared with LED egg candling.

Certain embodiments pertain to LSCI techniques that include one or more Fourier filtering techniques and/or one or more denoising algorithms to help mitigate image reconstruction artifacts resulting from chicken embryo body movement and background noise. In addition or alternatively, LSCI techniques may include a sliding window based temporal processing technique that can boost temporal resolution using as few as three raw speckle images in each temporal sliding window. These LSCI techniques can provide a comprehensive, non-invasive visualization of blood vessels of avian (e.g. chicken and quail) eggs both with high spatial and temporal resolution and demonstrated practical uses. These LSCI techniques can perform live view imaging of the avian egg's blood vessels network.

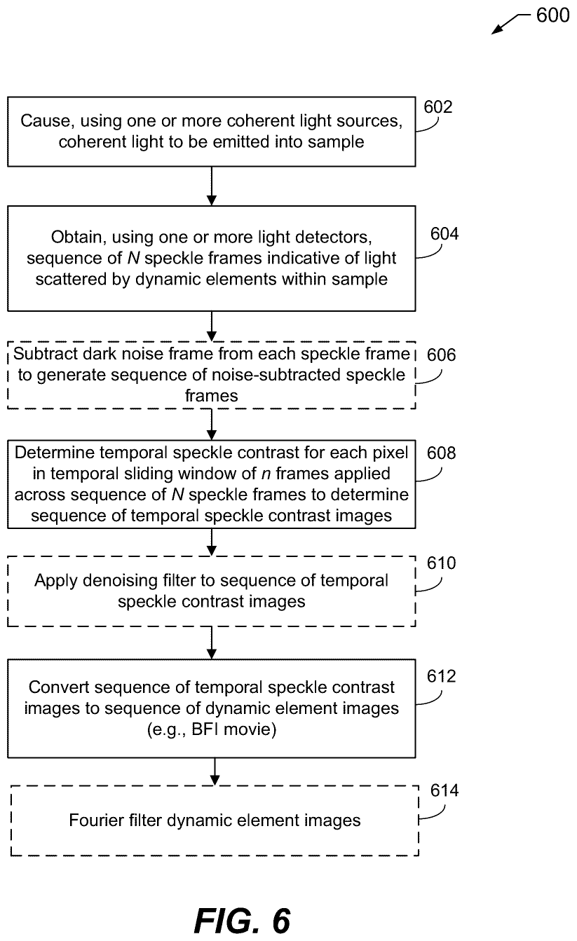

Certain embodiments pertain to LSCI methods that reconstruct a series of speckle contrast images or evaluate blood vessel metrics over time from different sets of raw speckle patterns using a sliding temporal window (also referred to herein as “sliding window-based temporal LSCI method”). These LSCI techniques may advantageously enhance temporal resolution. The sliding temporal window is generally a set of adjacent raw speckle patterns (e.g., two raw speckle images, three raw speckle images, four raw speckle images, etc.) from the raw speckle patterns captured by the image detection system during an exposure time. The sliding temporal window “slides” to use different sets of speckle patterns at different time periods within the exposure time. Each set of raw speckle patterns in the sliding temporal window is used to reconstruct a speckle contrast image and/or evaluate a blood metric at the corresponding time period. In some cases, the sliding temporal window overlaps sharing one or more speckle patterns. Sections II and III discusses results of using an implementation of the sliding window-based temporal LSCI method described in FIG. 6. Certain examples of sliding window-based temporal LSCI methods described herein may advantageously boost the temporal resolution of speckle images using as little as three raw speckle images in each temporal sliding window. In addition or alternatively, LSCI techniques may include one or more Fourier filtering procedures and/or one or more denoising procedures. Fourier filtering and denoising can advantageously mitigate image reconstruction artifacts that might result from chicken embryo body movement and background noise.

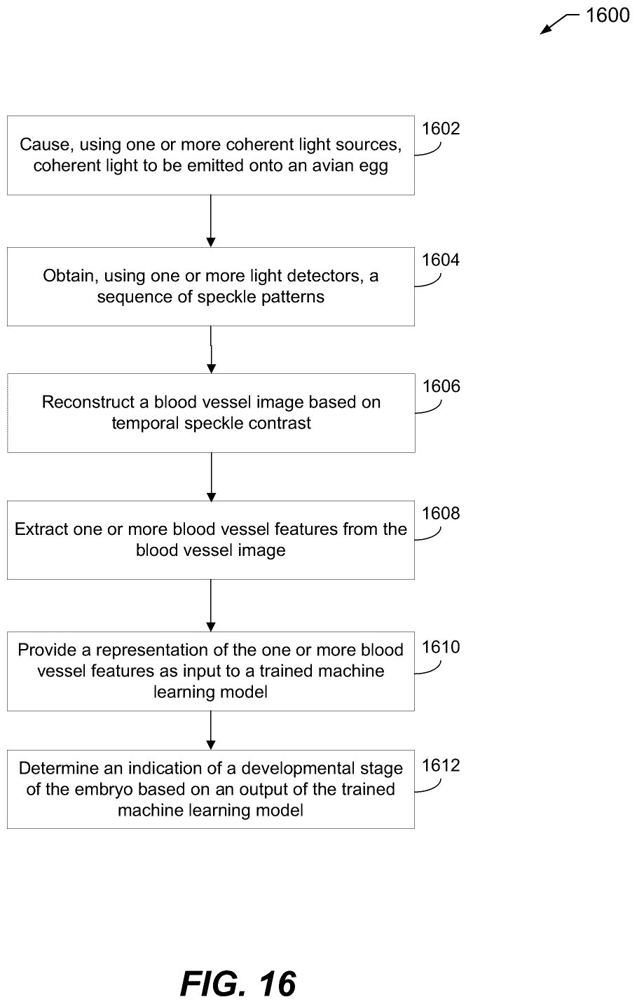

Certain embodiments pertain to LSCI techniques that can classify the developmental stage (also referred to as “staging”) of one or more embryos. For example, LSCI techniques may construct blood vessel images from speckle patterns and perform physical feature extraction on the blood vessel images. The feature extraction output can be input into a machine learning model (e.g., a deep neural network (DNN) such as a convolutional neural network) to determine the embryonic development stage across a plurality of different developmental stages (e.g., four different Hamilton-Hamburger (HH) stages). An example of such a technique is described with respect to FIG. 16. Using this example technique resulted in a developmental stage classification accuracy of 89% based on a dataset consisting of 220 blood vessel images, which surpassed the accuracy achieved with LED candling and human assessment. These results underscore the potential of using direct blood vessel images reconstructed by LSCI techniques or physical features extracted from these direct blood vessel images, such as vessel length, branches, area, and velocity distribution, for staging of avian embryos, which may eliminate the need for traditional methods that involve egg opening and that rely on morphological differences in the embryo body. In one implementation. In one implementation, an LSCI technique may be used for imaging chicken eggs blood vessels network between day-3 and day-5, and then feeding the blood vessel images into a convolutional neural network and predicting sex of the chicken embryo in its early development period based on output from the convolutional neural network.

### Drug Screening

Hypertension is a leading risk factor for cardiovascular disease and premature death. With more than 30% of adults worldwide affected by hypertension, extensive studies have been done on comparing different classes of treatments. An important class of drugs in this respect are the antihypertensive drugs belonging to the L-type calcium channel antagonist family, which include Nifedipine and Amlodipine. Nifedipine is sometimes used in conjunction with herbal remedies, while Amlodipine is commonly used for treatment due to its long half-life.

Since all vertebrate hearts including the human heart develop and function in the same way as the chick embryonic heart, evaluating the extraembryonic blood vessels in the CAM is considered a valid system for testing drugs in a preclinical trial. As discussed further herein, LSCI techniques have been employed to show chick embryonic heart response to the human anti-hyperintensive drugs, Amplodipine and Nifedipine. These and other drugs can be injected with a fine hypodermic needle through the eggshell ventral to the embryo. Certain LSCI techniques described herein can be used to visualize blood vessels and monitor blood flow noninvasively in a developing chick heart as a functional model for drug screening. These LSCI techniques may provide a rapid, cost-effective, and qualitative test system for screening drugs that affect the cardiovascular system. One advantage to these LSCI techniques is that they can be used to test heart function and vasculogenesis in a quantifiable non-invasive way. Presently, these drugs are tested preclinically on cardiac cells or organoids in vitro, which does not address the impact of the drugs on heart and vascular function. Some examples of drugs that may stand to benefit from using LSCI techniques in preclinical trials include: 1) heart drugs for hypertension, 2) Vitamin A/Retinoic acid and heart development (Vitamin A deficiency during pregnancy causes heart malformations in the fetus), 3) drugs affecting angiogenesis and 4) tumor vascularization and anti-angiogenic drugs. Angiogenesis, the formation and remodeling of blood vessels from existing ones, is key to the normal and disease conditions. Normal development of the embryo is dependent on angiogenesis while in adults angiogenesis is crucial for placenta formation, menstruation and wound healing. Angiogenesis plays a role in diseases such as: arthritis, diabetic retinopathy and tumor growth. Anti-angiogenic drugs such as Bevacizumab (Avastatin), Ramucirumab, and vitamin C are used as cancer therapies.

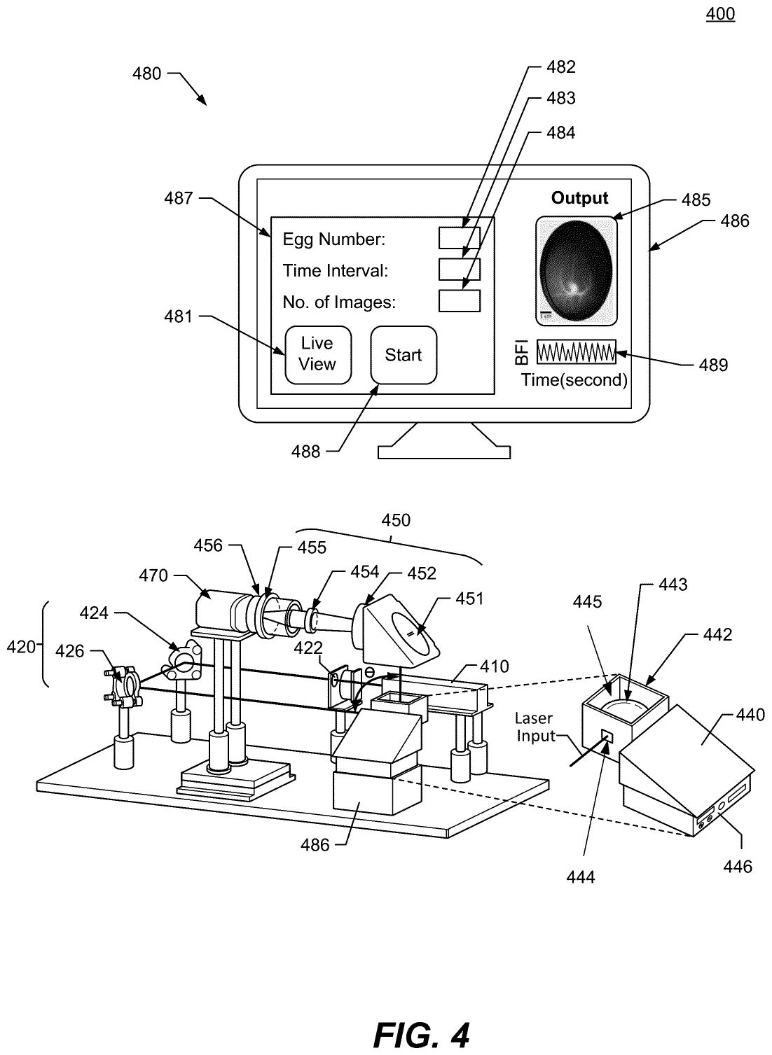

In some embodiments, LSCI systems (e.g., LSCI system 400 in FIG. 4) have an integrated incubator with one or more egg holders. In some cases, the LSCI systems may also employ automated data acquisition and analysis at chosen time points. The integrated incubator can be implemented for live imaging of the chick heart and evaluation of extraembryonic blood flow. These LSCI systems can be used in longitudinal studies of the embryonic heart and the drugs and other factors that can affect its function. These LSCI systems are non-invasive and do not alter the natural development of the chick embryo.

The LSCI system 400 shown in FIG. 4 was used to test two drugs that are commonly used to treat hypertension in humans: Nifedipine and Amlodipine. These drugs were injected through the shell ventral to chick embryos at a developmental HH stage between HH16-19. The needle was guided to the embryos by the LLSCI system in live view mode. The experimental data demonstrated a significant reduction in the chick's heart rate, blood flow in vessels within 5-15 min after Nifedipine or Amlodipine injection. For moderate Nifedipine concentrations, these parameters returned to initial values within 2-3 hours. Nifedipine was shown to be effective at a concentration ten times lower than amlodipine. The experimental data demonstrates that the LSCI system 400 in FIG. 4 was able to monitor and distinguish the chick heart's response to injected drugs from the same family.

### II. LSCI Systems

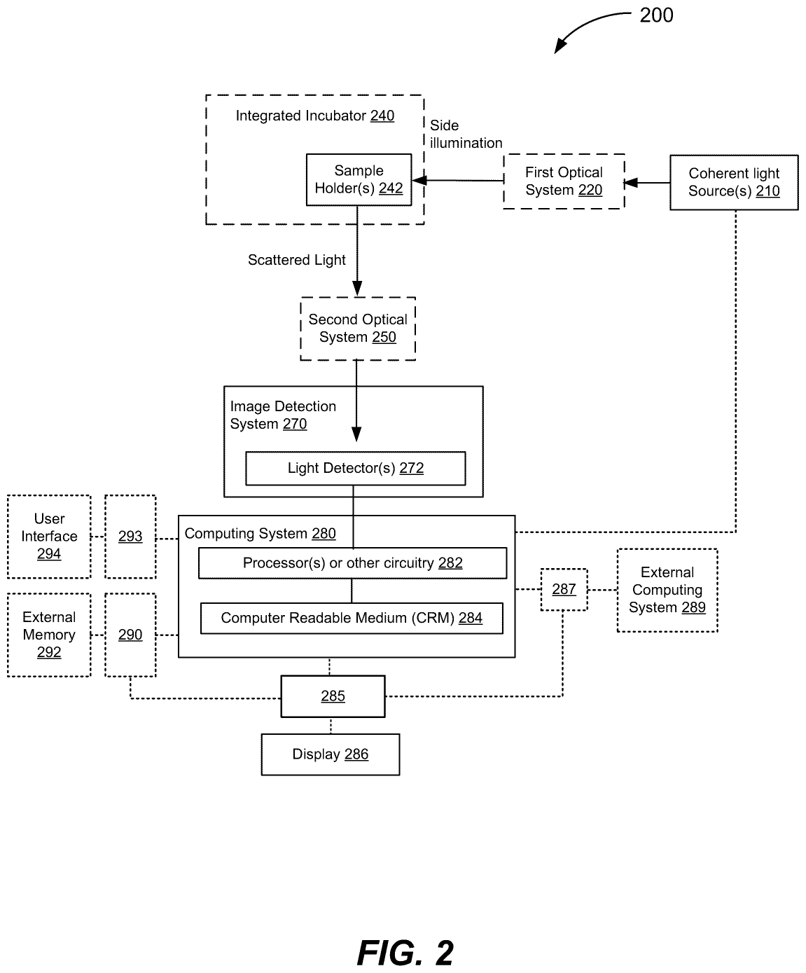

FIG. 2 is a simplified block diagram of components of an LSCI system 200, according to various embodiments. The LSCI system 200 includes one or more laser sources 210, one or more sample holders 242, and an image detection system 270 (e.g., a camera) including one or more light detectors 272 that can be used to image raw speckle patterns (frames). Some examples of types of light detectors that may be used include complementary metal oxide semiconductor (CMOS) image sensors, charge-coupled devices (CCD), and other similar devices.

According to certain embodiments, an LSCI system includes one or more laser sources. In one embodiment, an LSCI system includes a near-infrared laser source. In some cases, an LSCI system includes a laser source that can provide illumination having a single wavelength. For example, a laser source may be an 852 nm near-infrared laser of up to 230 mW. Note that laser light may be emitted continuously or may be pulsed.

In certain implementations, such as those that may be used in longitudinal studies, the LSCI system 200 optionally (denoted by dashed line) includes an integrated incubator 240. The integrated incubator 240 may have a tray including the one or more sample holders 242. The tray may be of various formats such as a linear tray, a rectangular tray, a circular tray, a concentric circle tray, etc. In one implementation, the tray includes multiple sample holder and is configured to be translated or rotated (e.g., tray attached to translational/rotation stage) to position the tray such that side illumination is incident one sample holder at each image acquisition and then moved the tray translated/rotated to position another sample holder for side illumination during another image acquisition. The tray may have any suitable number of sample holders. According to one aspect, a sample holder 242 is a 3-D printed structure such as may be printed by a 3-D resin printer.

In one implementation, the integrated incubator 240 includes a chamber for maintaining one or more environmental conditions (e.g., temperature, humidity, and/or air flow). The integrated incubator 240 may include an aperture (e.g., aperture 444 in FIG. 4) on one side that is left open or covered by a transparent material (e.g., transparent tape). The aperture may be of any suitable size and shape (e.g., circle, rectangle, etc.) for allowing illumination from the one or more laser sources 210 to illuminate the sample (e.g. egg) from the one side. For example, the aperture (e.g., aperture 444 in FIG. 4) may be a 1.4 mm×1.4 mm square aperture. The integrated incubator 240 may also have an opening on another side (e.g., opening 445 in FIG. 4) that is covered with a transparent plastic window to allow light scattered by the sample being imaged to propagate to the one or more light detectors 272.

Optionally, LSCI system 200 also includes a first optical system 220 and/or a second optical system 250. The first optical system 220 has one or more optical components (e.g., one or more of beam splitters, lenses, optical fibers, mirrors, apertures, or other optics) configured to propagate the laser illumination from the one or more laser sources 210, or block (shutter) the illumination from propagating, in a first direction onto the sample (sometimes referred to herein as “side illumination). The side incident light may be scattered by elements within the sample. The second optical system 250 includes one or more optical components (e.g., one or more of beam splitters, lenses, optical fibers, mirrors, apertures, or other optics) configured to collect light from a second direction that is scattered by the sample and provide the light onto the one or more light detectors 272. In one example, the second optical system 250 includes an optical shutter (e.g., a digitally-controller optical shutter) for blocking the one or more laser sources 210 when the image detection system 270 is not recording or when the image detection system 270 is recording speckle frames for determining a dark noise frame. In some implementations, the first optical system 220 and/or a second optical system 250 may be integrated into other components of LSCI system 200. For example, the first optical system 220 may be part of the one or more laser sources 210 and/or the second optical system 250 may be part of the image detection system 270.

Some examples of arrangements and types of optical components that can be included in first optical system 220 are those of first optical systems 320 and 420 in FIGS. 3 and 4 respectively. Some examples of arrangements and types of optical components that can be included in second optical system 250 are those of second optical systems 350 and 450 in FIGS. 3 and 4 respectively. The types of optical components and arrangements of the optical components of the first and second optical systems in FIGS. 3 and 4 are examples. It would be understood that in other implementations, other arrangements and optical components can be used.

The LSCI system 200 provides side illumination to the sample being imaged in a first direction that is at an angle from a second direction of light scattered by the sample and collected by the second optical system 230. In one aspect, the angle may be about 90 degrees. In another aspect, the angle may be between 45 degrees and 135 degrees. In another aspect, the angle may be about 180 degrees.

According to various implementations, the image detection system 270 includes a camera such as a high-resolution camera. An example of a high-speed camera that can be used is the CS126CU camera sold by Thorlabs. Various exposure times and frame rates may be used according to various aspects. In one aspect, the exposure time may be in a range of 0.1 ms and 10 ms. In another aspect, the exposure time may in a range above 10 ms. In one aspect, the frame rate may be in a range of 1 FPS to 50 FPS. In another aspect, the frame rate may in a range above 50 FPS for higher temporal resolution imaging. In one example, an exposure time is about 10 ms and a frame rate is about 21 frames/second. When using the CS126CU camera, a speckle image may be captured during each exposure time with a resolution of about 3000×4096 pixels, a pixel pitch size of 3.45×3.45 μm, and a speckle size of about two pixels per speckle. The CS126CU camera has a quantum efficiency of approximately 20% at a laser wavelength of 852 nm. In another example, camera Basler boost R boA4500-45 cm with 1.3” sensor format, 20 mega-pixels resolution and 45 FPS frame rate can be used.

The LSCI system 200 also includes a computing system 280 having one or more processors and/or other circuitry 282 and a non-transitory computer readable medium (CRM) 284 in electrical communication with the processor(s) or other circuitry 282. The computing system 280 is in electrical communication with the one or more light detectors 272 to receive image data. Some examples of types of processors that can be used include one or more of a general purpose processor (GPU), an application-specific integrated circuit, a programmable logic device (PLD) such as a field-programmable gate array (FPGA), or a System-on-Chip (SoC) that includes one or more of a central processing unit (CPU), application-specific integrated circuit, PLD as well as a memory and various interfaces. Optionally, the computing system 280 may also be in electrical communication with the one or more laser source(s) 210 to send control signals for controlling operations.

The processor(s) or other circuitry 282 of the computing system 280 and, additionally or alternatively, other external processor(s) (e.g., a processor of the external computing system 289) can execute instructions stored on non-transitory computer readable media (e.g., internal non-transitory CRM 284 or optional external memory device 292) to perform operations of the LSCI system 200. For example, the processor(s) or other circuitry may execute instructions to perform operations of an LSCI method. As another example, the processor(s) or other circuitry may send control signals to activate the one or more laser source(s) 710 and/or may send control signals to activate the image detection system 270 to record raw speckle patterns.

The LSCI system 200 includes an internal computer readable medium 284 and optionally includes an external memory device 292. The internal computer readable medium 284 can include a non-volatile memory array for storing processor-executable code (or “instructions”) that is retrieved by one or more processors or other circuitry 282 to perform various functions or operations described herein for carrying out various logic or other operations on the image data. The internal computer readable medium 284 also can store raw image data, processed image data, and/or other data. In some implementations, the internal computer readable medium 284 or a separate memory device can additionally or alternatively include a volatile memory array for temporarily storing code to be executed as well as image data to be processed, stored, or displayed. In some implementations, the computing system 280 itself can include volatile memory and in some instances also non-volatile memory.

LSCI system 200 also includes an optional communication interface 285 and a display 286 in communication with the communication interface 285. The computing system 280 may be configured or configurable to output raw data, processed data such as image data, and/or other data over the communication interface 285 for display on the display 286. Optionally (denoted by dashed lines), the LSCI system 200 may further include one or more of a first communication interface 287, an external computing system 289 in communication with the first communication interface 287, a second communication interface 290, an external memory device 292 in communication with the second communication interface 290 for optional storage of data to the external memory device 292, a third communication interface 293 in communication with a user interface 294 for receiving input from an operator of the LSCI system 200. The optional user interface 294 is in electrical communication with the LSCI system 800 through the third communication interface 293 to be able to send a control signal to the computing system 280 based on input received at the user interface 294.

In some implementations, the computing system 280 may be configured or configurable (e.g., by a user) to: (i) process raw speckle patterns and other raw data such as traces of blood vessel metrics as a function of time, (ii) output raw data, processed data, and/or other data over communication interface 285 to display 286, (iii) output raw data as well as processed image data and other processed data over first communication interface 287 to external computing device or system 289, (iv) output raw image data as well as processed image data and other data over second communication interface 290 for storage on external memory device or system 292, and/or (v) output raw image data as well as processed image data over a network communication interface for communication over an external network (for example, a wired or wireless network). Indeed in some implementations, one or more of operations of an LSCI method can be performed by an external computing device. The computing system 280 may also include a network communication interface that can be used to receive information such as software or firmware updates or other data for download by the computing device.

### Dual-Channel LSCI System

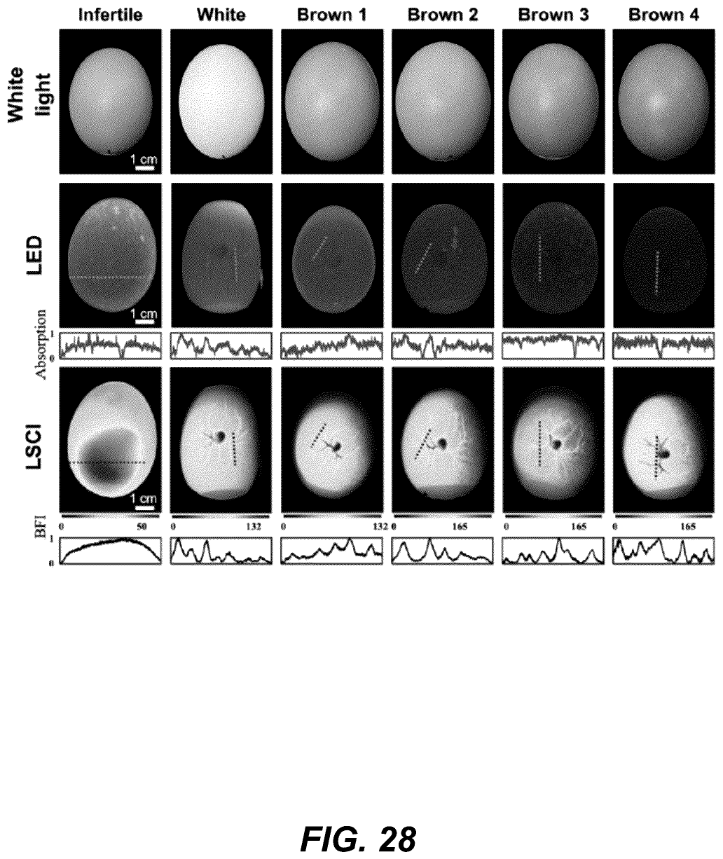

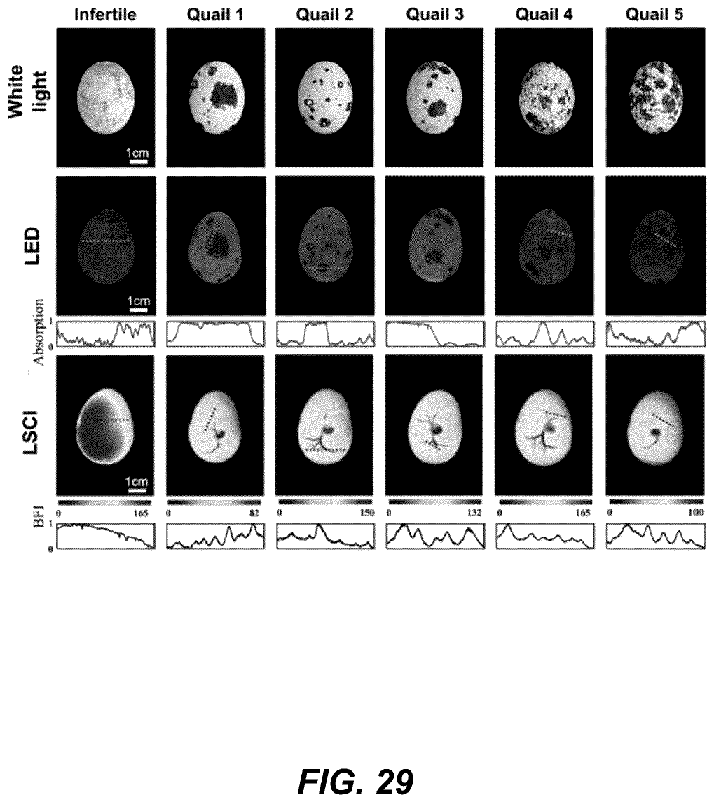

According to one embodiment, a LSCI system is a dual-channel system that includes components for brightfield transmission imaging and laser speckle imaging. This dual-channel LSCI system includes one or more broadband light sources that provide broadband illumination to one side of the sample (e.g., egg) where light is collected from an opposing side at one or more light detectors to capture brightfield images. This dual-channel LSCI system integrates the one or more broadband light sources using the same detection path as used to collect scattered light from coherent side illumination. This dual-channel LSCI system can be used to compare speckle contrast images based on laser speckle patterns captured under coherent illumination with brightfield images captured from through transmission under broadband illumination. An example of such a dual channel LSCI system is dual-channel LSCI system 300 shown in FIG. 3. FIGS. 28 and 29 show comparison of laser speckle images with bright field images captured by dual-channel LSCI system 300 shown in FIG. 3. Brightfield images captured using through transmission under broadband illumination from light emitting diodes (LEDs) is sometimes referred to herein as “LED egg candling.” The comparison shows that laser speckle imaging is more resistant to variations in eggshell color and shade as compared with LED egg candling.

FIG. 3 is a simplified block diagram of components of the dual-channel LSCI system 300 that can be used for both brightfield transmission imaging and laser speckle imaging, according to an embodiment. For the sake of brevity, the prior discussion of similar or analogous elements with regards to FIG. 2 may be assumed to be equally applicable, unless indicated otherwise in the following discussion, to the similar or analogous counterparts of those elements in FIG. 3 that share the same last two digits in their respective callouts as in FIG. 2. The brightfield transmission imaging and laser speckle imaging modes share the same detection configuration where the sample 343 is imaged by the same camera 370 and light emitted from the sample 343 is collected by the same second optical system 350.

The dual-channel LSCI system 300 includes a laser source 310 that can provide coherent laser light, a first optical system 320, a broadband light source 332, and an image detection system including a second optical system 350 and a camera 370. The first optical system 320 provides the laser light from laser source 310 in a first direction onto a side of the sample 343 (sometimes referred to herein as “side illumination”). In some cases, laser source 310 may be a single frequency continuous wave 852 nm laser (e.g., DL852-300-SO laser sold by Spectra-Physics) with an output power of 230 mW. In one implementation, the laser source 310 can provide laser light and the first optical system 220 propagates the laser light for illuminating samples (e.g., avian eggs) from one side with a laser beam diameter of 5 mm. Other laser sources may be used according to other implementations.

The brightfield transmission imaging components include a broadband light source 332 and a coupling lens 334 inserted after the broadband light source 332 to collimate the light onto the sample 343. In an alternative embodiment, the brightfield components (e.g., broadband light source 332 and lens 334) of the dual channel LSCI system 300 shown in in FIG. 3 may be omitted such that the system functions only in laser speckle imaging mode. In one implementation, the broadband light source 332 is a 35 nm bandwidth incoherent light source with an output power of 480 mW.

The dual-channel LSCI system 300 also includes a sample holder 342 that can be used to hold a sample 343 (e.g., egg) being imaged. In FIG. 3, the brightfield transmission imaging components of the dual-channel LSCI system 300 are configured to illuminate the sample 343 from underneath, in a direction perpendicular to a horizontal plane at the platform. During operation, the sample 343, which is a chicken egg in this instance, may be disposed on the sample holder 342 (e.g., egg holder) in a horizontal position. The sample holder 341 includes a surface with an elliptical shape to fit in and stabilize the egg position during imaging.

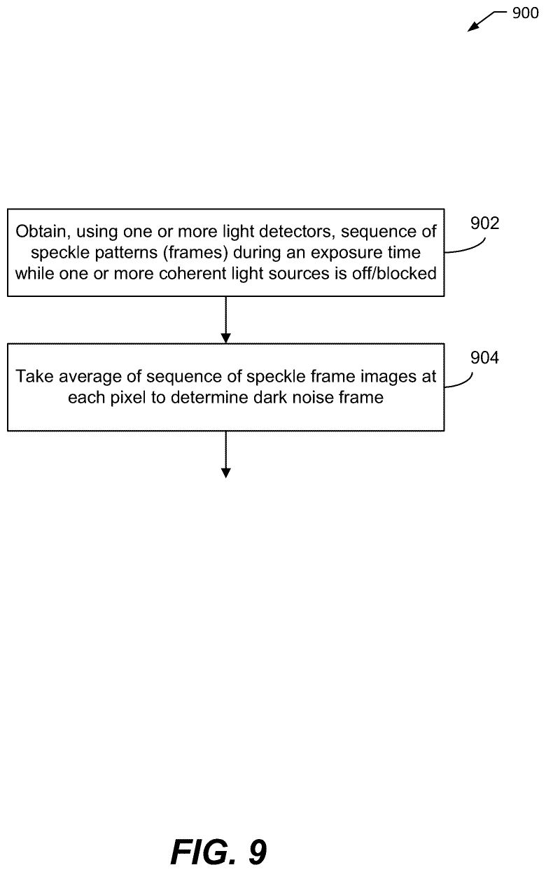

The first optical system 320 includes an optical shutter 322 placed after the laser source 310 to block the laser source 310 when the camera 570 is not recording or when speckle patterns are being recorded to determine a dark noise frame. A discussion of an example procedure for collecting speckle patterns for a dark noise frame is discussed with reference to FIG. 9. The optical shutter 322 may be digitally controlled in one implementation. The first optical system 320 also has a pair of reflective mirrors including a first reflective mirror 324 and a second reflective mirror 326 in a face-to-face 90 deg configuration to direct the laser light in a first direction onto the sample 343 for side-illuminating the sample (e.g., chicken egg). The first optical system 320 also includes a linear polarizer 328 prior to the sample 343 to remove polarized light that might cause speckle disruptions. In other implementations, the linear polarizer 328 may be omitted.

The scattered light exiting the sample 343 is collected from a second direction (e.g., from the top view in FIG. 3) by the second optical system 350, which provides collected light onto a camera 370. The first direction of the optical path of the laser light being provided onto the sample 343 is at an angle, e, (e.g., about 90 degrees) from the second direction of the optical path of light collected by the second optical system 350. In one aspect, the angle may be about 90 degrees. In another aspect, the angle may be between 45 degrees and 135 degrees. In another aspect, the angle may be about 180 degrees. Other angles may be used in other implementations.

The second optical system 350 includes a third mirror 351 for changing the propagation orientation from vertical to horizontal. The second optical system 350 also includes a first aperture 352 inserted next to the third mirror 351 to control the region-of-interest of the field-of-view and to filter out undesired stray light. The second optical system 350 also has filters 353 including a low-pass filter (e.g., FESH0700 low-pass filter sold by Thorlabs) and a band-pass filter (e.g., FBH850-10 band-pass filter sold by Thorlabs) of the same or nearly the same wavelength (e.g., 850±5 nm) as the laser source 310. The low-pass filter is placed in the optical path during brightfield transmission image acquisition to only let visible light pass through. The band-pass filter is placed in the optical path during speckle pattern image acquisition. The second optical system 350 also includes an imaging lens 354, which may be tunable, and a second aperture 355, which is adjustable. In one example, the imaging lens 354 may have a 50 mm focal length (e.g., #86-574 lens sold by Edmund Optics). The second optical system 350 also includes an optical shutter 356 placed in front of the camera 370 that can be used to switch on/off raw speckle pattern (frame) acquisition. The optical shutter 356 may be digitally controlled according to one aspect.

In one aspect, certain LSCI systems may be configured, or are configurable, to set the speckle size of the speckle patterns. For example, an aperture placed in a pupil plane of a lens in front of the camera may be adjusted to set the speckle size. As an illustrated example, the adjustable aperture 355 shown in FIG. 3 is placed in the pupil plane of the lens 354 in front of the camera 370 to enable adjustment of the speckle size of the light field. In one case, the magnification of the image detection system of dual-channel LSCI system 300 is 0.2 and the equivalent numerical aperture is 0.03 such that the speckle size is about two pixels per speckle. In one aspect, the size of the aperture 355 near to pupil plane of the imaging lens 354 and the magnification of the second optical system 350 may be selected to ensure field-of-view coverage of the entire sample 343 and the speckle size on the camera plane to be larger than two pixels. In one case, an LSCI system may be configured to have a speckle size of about two pixels per speckle. In another case, an LSCI system may be configured to have a speckle size of about three pixels per speckle.

Returning to FIG. 3, the dual-channel LSCI system 300 includes a camera 370 that can record the emitted light from the sample 343. In some cases, the camera 370 is an RGB camera that can operate in RBG mode during brightfield transmission image acquisition and in monochromatic mode during speckle pattern acquisition. For example, the camera 370 may be the CS126CU RGB camera sold by Thorlabs with an exposure time ranging from 0.03 ms to 14.7 s. The CS126CU RGB camera has a camera resolution is 3000×4096 pixels, with a pixel pitch size of 3.45×3.45 μm. During brightfield transmission image acquisition, the camera may operate in an RGB mode with an exposure time set in the range 0.5 s to 3.5 s and during speckle pattern acquisition, the camera may operate in the monochromatic mode with an exposure time set at T=10 ms and at a speed of 21 frame per second.

In one example, the broadband light source 332 may include a 530 nm LED (e.g., M530L4 LED sold by Thorlabs) delivering a 35 nm bandwidth incoherent light with an output power of 480 mW. The incident light on the sample 343 may be absorbed by its components (e.g., yolk, embryo, CAM, and eggshell of an egg) and the transmission image collected by the camera 370. For instance, when imaging an egg, the green light from the broadband light source 332 may propagate inside the egg. The through transmission light and emission exiting the egg can be collected by the second optical system 350 and imaged onto the camera 370. During brightfield transmission imaging, the red channel of the camera 370 captures a fluorescence image while the green channel captures an absorption image.

In various embodiments, a computing system of an LSCI system includes a display (e.g., display 386 in FIG. 3 and display 486 in FIG. 4) with GUI elements (e.g., GUI elements 387 in FIG. 3 and GUI elements 487 in FIG. 4) that can be used to enter user settings for controlling imaging and/or to provide output of processed data. For example, the user settings may be used to control the frame rate and exposure time of the camera during recording of speckle patterns. As another example, the user setting may be used to control turning on/off the optical shutter in front of the laser source to block/unblock laser light.

In FIG. 3, dual-channel LSCI system 300 also includes a computing system 380 with a display 386 including a plurality of graphical user interface (GUI) elements 387 for entering user settings for controlling imaging. The GUI elements 387 include an icon 381 for initiating a “live view mode.”

As used herein, a “live view mode,” generally refers to an operational mode of an LSCI system that enables viewing of a time varying series (sequence) of images (e.g., a movie) of dynamic components of a sample (e.g., blood vessels, beating heart of avian embryo, etc.) or a time varying series of blood vessel metrics (e.g., heartbeat over time) of a sample. For example, live view mode can enable the ability to view a sequence of extraembryonic blood vessel images (e.g., movie of blood vessels over time) in an avian egg. An example of a procedure for determining a time varying series of blood vessel images based on speckle patterns captured by an image detection system of a LSCI system is described with respect to FIG. 6 below. In the example procedure, each blood vessel image is reconstructed from a corresponding overlapping set of speckle frames (e.g., a set of three speckle frames, a set of four speckle frames, a set of two speckle frames, etc.) from shifting strides of the sliding window over time from the sequence of speckle patterns captured by the image detection system during an exposure time. The time increment between images in the movie can be any suitable time period (e.g., one image/second, two images/second, three images/second, etc.). In one example, a blood vessel image is provided every second in the movie where each image is reconstructed from a set of speckle frames.

The GUI elements 387 also include elements that enable a user to enter settings for image acquisition on one or more samples. For example, GUI elements 387 include a first field 382 for entering a sample identifier, a second field 383 for entering a recording interval in minutes, and a third field 384 for entering the number of speckle patterns to capture during the recording interval. The GUI elements 487 also includes a start button 388 for initiating image acquisition process. In one implementation, triggering the start button will initiate an automated process using the values entered into first field 382, second field 383, and third field 384, and that may not necessarily require additional input from a user. The display 386 also includes output including an image 385 (e.g., BFI map) of the blood vessels of sample 343 being imaged and a graph 389 of blood flow dynamics (e.g., BFI) over time (e.g., over 5 seconds).

The computing system 380 also includes one or more processors (e.g., processors and/or other circuitry 282 in FIG. 2) and a non-transitory computer readable medium (e.g., CRM 284 in FIG. 2) in electrical communication with the processor(s) or other circuitry. The computing system 380 is in electrical communication with optical shutter 356 and/or the one or more light detectors 372 to send control signals. Optionally, the computing system 380 may also be in electrical communication with the laser source 310, optical shutter 322, and broadband light source 332 to send control signals. The processor(s) or other circuitry of the computing system 380 and, additionally or alternatively, other external processor(s) can execute instructions stored on non-transitory computer readable media to perform operations of the dual-channel LSCI system 300.

The internal computer readable medium of the computing system 280 can include a non-volatile memory for storing processor-executable code (or “instructions”) that is retrieved by one or more processors or other circuitry to perform various functions or operations described herein for carrying out various logic or other operations on the image data. The internal computer readable medium also can store raw image data, processed image data, and/or other data. In some implementations, the internal computer readable medium or a separate memory device can additionally or alternatively include a volatile memory array for temporarily storing code to be executed as well as image data to be processed, stored, or displayed. In some implementations, the computing system 380 itself can include volatile memory and in some instances also non-volatile memory.

Dual-channel LSCI system 300 may also include one or more communication interfaces in communication with various devices such as an external computing system, an external display, ana external memory device, etc. The computing system 380 may also include a network communication interface that can be used to receive information such as software or firmware updates or other data for download by the computing device.

In some implementations, the computing system 380 may be configured or configurable (e.g., by a user) to: (i) process raw speckle patterns and other raw data such as traces of blood vessel metrics as a function of time, (ii) output raw data, processed data, and/or other data to display 386, (iii) output raw image data as well as processed image data and other processed data to an external computing device, (iv) output raw image data as well as processed image data and other data for storage on an internal or external memory device, and/or (v) output raw image data as well as processed image data over a network communication interface for communication over an external network.

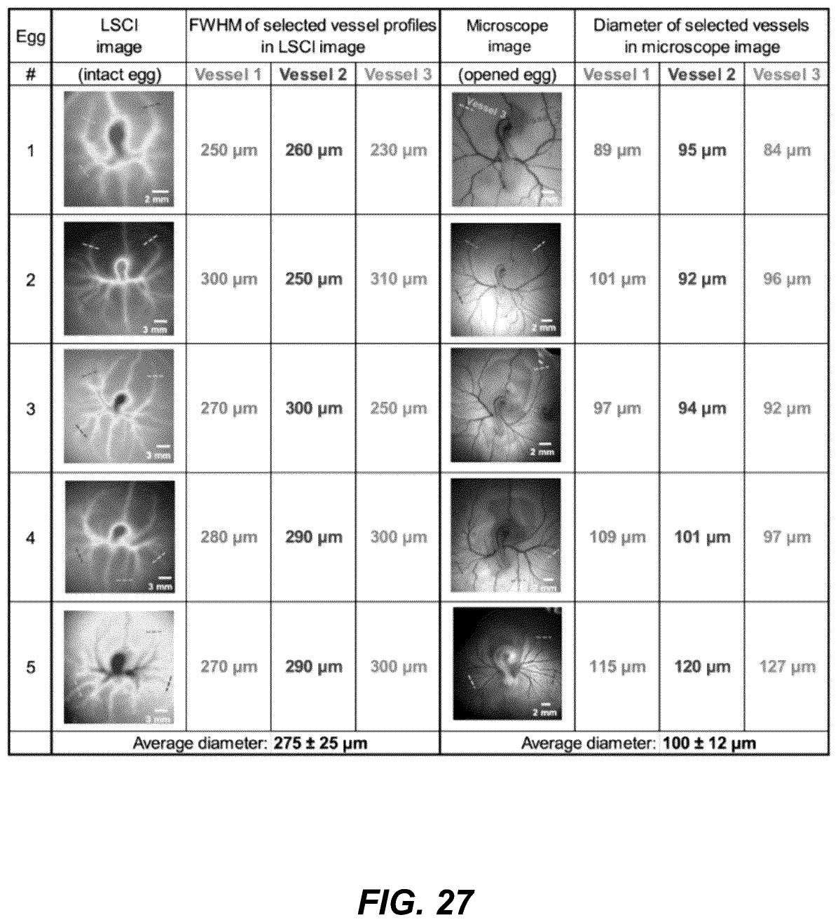

The LSCI system 300 described in connection with FIG. 3 was used to determine the diameters of the smallest blood vessels of five eggs. For comparison, the eggs were opened and protective membrane removed, and the embryo was imaged with a conventional microscope. FIG. 27 is a table of the smallest blood vessel diameters for five eggs by LSCI system 300 described in connection with FIG. 3 and using a conventional microscope for comparison. Across five different eggs, the LSCI system 300 was used to measure average full width at half maximum (FWHM) across the five eggs to be 275+25 μm. In the corresponding microscope images, the average diameter of the selected blood vessels was measured across the five eggs to be 100±12 μm. This set out the smallest blood vessel diameter detectable by the LSCI system to be 100 μm.

The LSCI system 300 described in connection with FIG. 3 was used to image six chicken eggs at day 4 of incubation. FIG. 28 is a table of photographic images of six chicken eggs, brightfield transmission images of the six chicken eggs, and images of the six chicken eggs generated by LSCI system 300 according to an embodiment. The top row of images are photographic images of the eggs showcasing the coloration of the shells. The middle row of images are brightfield transmission images. The blood vessels in the white egg or light brown eggs (Brown 1 and Brown 2) can be observed but they cannot be observed in dark brown eggs (Brown 3 and Brown 4). The absorption line profiles are calculated as the inversion of normalized green channel intensity of the RGB images. The eggshell crack positions have lower absorption and show up as dips in the line profiles. The bottom row of images was generated by LSCI system 300 described in connection with FIG. 3. In these images, the blood vessels can be effectively observed for all egg types. Note that the first egg is infertile, displaying no signs of an embryo. The BFI line profiles are normalized to between 0 and 1.

For the infertile egg, only the movement of the yolk was observed, characterized by a relatively low BFI. In contrast, both white and brown fertile eggs exhibited distinct visibility of blood vessels surrounding the embryo and shared similar BFI values. Notably, there was no disparity in LSCI imaging quality across white egg, dark brown egg (e.g., Brown 3), and light brown egg (e.g., Brown 1), as shown in the BFI line profiles in FIG. 28 where the position of the blood vessels can be identified by the peaks consistently. These observations show that the color of the eggshell has no significant impact on the LSCI imaging results. Since the eggshell remains largely static (non-moving), the speckle pattern originating from the eggshell is stationary (static speckle pattern) and is effectively filtered out during temporal speckle contrast calculations. Conversely, as there are blood flow movements in the vessels of the chicken embryo, the speckle pattern undergoes changes over time (resulting in a dynamic, moving speckle pattern) and is captured by higher BFI values through temporal LSCI computations. Consequently, eggshell cracks were apparent with the brightfield transmission imaging method (see dip positions in the absorption line profiles in but were absent with the LSCI images.

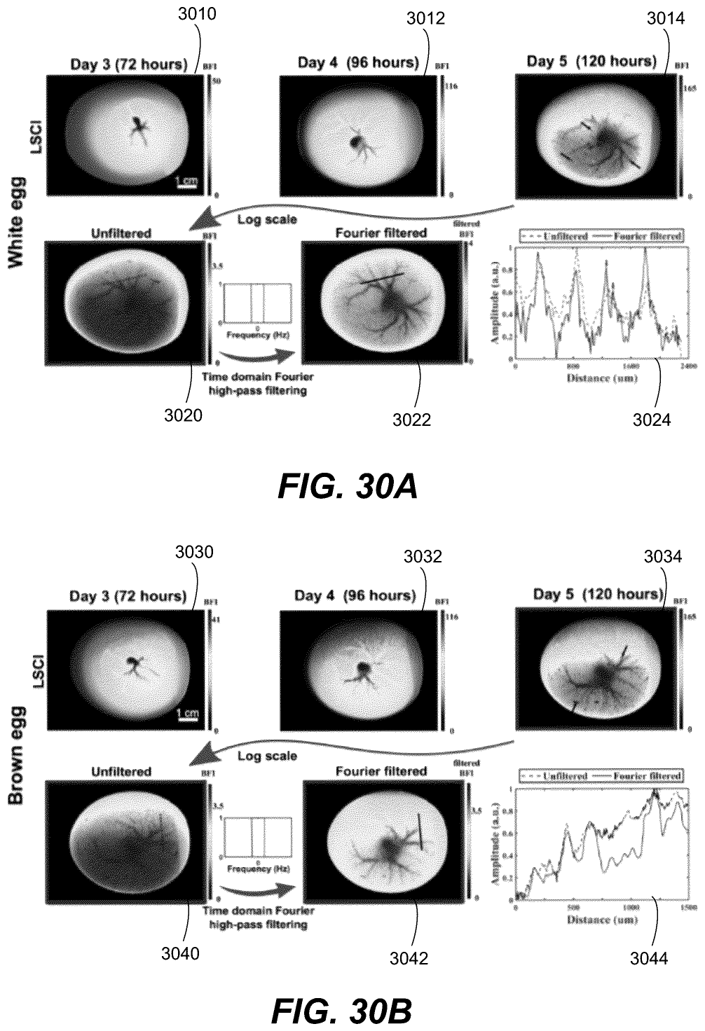

The LSCI system 300 described in connection with FIG. 3 was used to image six quail eggs at day 4 of incubation. FIG. 29 is a table of photographic images of six quail eggs, brightfield transmission images of the six quail eggs, and images of the six quail eggs generated by LSCI system 300 according to an embodiment. In the LSCI images generated by the LSCI system 300, the blood vessels can be readily observed on white regions of the eggshell, whereas they are not visible on eggshell regions with black spots. Opposed to the brightfield transmission imaging method, the LSCI method is not influenced by the color, shade, or cracks of the eggshell. The LSCI system 300 described in connection with FIG. 3 was used to track blood vessel network development. FIG. 30A depicts images of white eggs across three incubation days: day 3 (72 hours), day 4 (96 hours), and day 5 (120 hours). FIG. 30B depicts images of brown eggs across three incubation days: day 3 (72 hours), day 4 (96 hours), and day 5 (120 hours). Between day 3 and day 5, both the white eggs and the brown eggs exhibit a progressive expansion of the vessel network and an increase in the BFI. By using Fourier filtering, a significant improvement can be achieved in imaging the chicken embryo blood vessel network.

The LSCI system 300 described in connection with FIG. 3 was used to monitor the blood flow in a chicken egg non-invasively at day 4.74 (114 hours) of incubation. To monitor the blood flow variations as a function of time, the LSCI system 300 of FIG. 3 was used to record N=300 speckle pattern images and a sliding window in the time domain over n=3 adjacent speckle pattern images to create a series of blood flow reconstructions over time. The camera operated at 21 frames per second (FPS), resulting in a movie of the chicken embryo's blood flow distribution map at 21 images per second. The rectangular sliding window in the time domain acts as a 7 Hz low-pass filter in the frequency domain, whose temporal Fourier transform is a Sinc function.

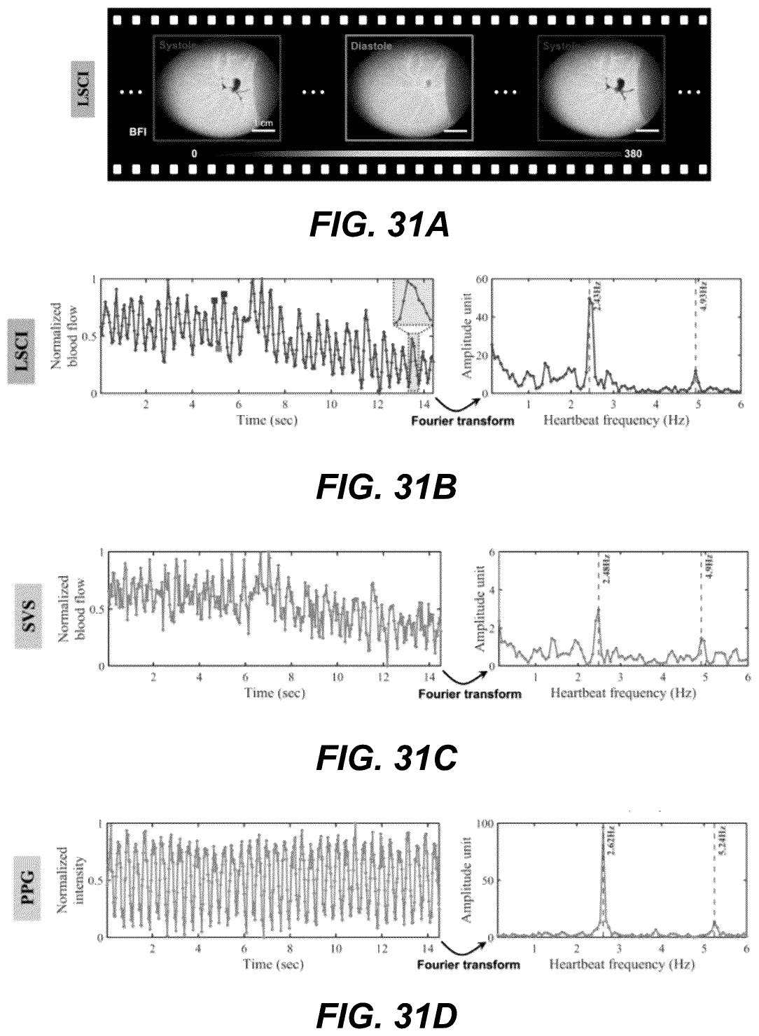

FIG. 31A depicts three snapshots from the LSCI movie. Each snapshot corresponds to a specific time of the cardiac cycle of the chicken egg, boxes for time indication. The blood flow at different periods of a cardiac cycle can be observed. During systole, the heart contracts, propelling blood into the arteries, leading to high BFI. In contrast, during diastole, the heart relaxes and refills with blood, causing a reduction in BFI within the arteries.

FIG. 31B depicts a plot of the heartbeat signal of normalized blood flow and a plot of the heartbeat frequency determined using the LSCI system 300 in FIG. 3, according to an embodiment. The averaged BFI value within the center cropped region calculated from each image of the movie in FIG. 31A. The heart rate was calculated by Fourier transforming the blood flow signal. A hamming window was applied before the Fourier transform to avoid spectral leakage. The Fourier amplitude peak centered around freqHR=2.43 Hz corresponds to the heart rate amplitude peak of the embryo. There was also a Fourier peak centered at 4.9 Hz corresponding to the second harmonic of the heart rate pulsations of the embryo.

FIG. 31C depicts a plot of the heartbeat signal of normalized blood flow and a plot of the heartbeat frequency using the speckle visibility spectroscopy (SVS) method. In SVS, instead of calculating the blood flow from the temporal speckle contrast of three adjacent speckle pattern images, the blood flow value at one-time point was calculated from the speckle contrast of each speckle pattern image, with each camera pixel acting as a sample point. SVS results were slightly noisier.

FIG. 31D depicts a plot of the heartbeat signal of normalized blood flow and a plot of the heartbeat frequency taken by opening up the egg and recording light microscopy images of the embryo. The intensity fluctuations here reflect absorption changes due to the embryo's cardiac circulation. Blood flow measurements closely resemble those obtained using the LSCI or SVS methods, but with an enhanced signal quality, attributable to the direct access to the embryo achieved by opening the egg.

### LSCI System With Integrated Incubator

In certain embodiments, an LSCI system includes an integrated incubator that can be employed when live imaging of dynamic elements of a sample and during longitudinal studies. For example, an LSCI system with an integrated incubator can be used for live imaging of a chick heart and extraembryonic blood flow, and can be used in longitudinal studies of the embryonic heart and the drugs and factors that can affect its function. Such an LSCI system with an integrated incubator can perform non-invasive imaging and does not significantly alter the natural development of the chick embryo.

FIG. 4 is a simplified block diagram of components of an LSCI system 400 with an integrated incubator 540, according to embodiments. For the sake of brevity, the prior discussion of similar or analogous elements with regards to FIG. 2 may be assumed to be equally applicable, unless indicated otherwise in the following discussion, to the similar or analogous counterparts of those elements in FIG. 4 that share the same last two digits in their respective callouts as in FIG. 2.

The LSCI system 400 includes a laser source 410 that can provide coherent laser light, a first optical system 420, and an image detection system including a second optical system 450 and a camera 470. The first optical system 420 provides the laser light from laser source 410 in a first direction onto a side of the sample 443 (sometimes referred to herein as “side illumination”). In one example, the image detection system has a magnification of 0.2 and a numerical aperture of 0.03. In some cases, the laser source 410 is configured to provide illumination having a single wavelength. In one aspect, the laser source 410 is a single frequency continuous wave 852 nm laser (e.g., DL852-300-SO laser sold by Spectra-Physics) with an output power of 230 mW. Other laser light sources may be used in other implementations.

The LSCI system 400 includes an integrated incubator 440 that can be used, for example, in longitudinal studies. The integrated incubator 440 includes an enclosed chamber 441 for maintaining one or more environmental conditions (e.g., temperature, humidity, and/or air flow). The integrated incubator 440 may include one or more devices (e.g., a heater, a fan, and/or egg turning device) to control the environmental conditions and control devices (e.g., knobs) to enter settings (e.g., temperature and humidity settings) for the devices.

The chamber 441 is configured to receive one or more sample holders, for example, a tray of sample holders. According to one implementation, the chamber 441 and/or the one or more sample holders may be 3-D printed (e.g. printed using resin 3D printer made by Anycubic). At the instant of the illustrated example shown in FIG. 4, the sample holder 442 with sample 443 (e.g., egg) has been taken out of the chamber 441 and positioned for image acquisition. The sample holder 442 may include a surface with an elliptical shape to fit in and stabilize the egg position during imaging. The chamber 441 also includes an aperture 444 that is either left open or covered by a transparent material (e.g., transparent tape). During image acquisition, sample holder 442 is positioned such that laser light from the laser source 410 passes through the aperture 444 to the sample 443 for side illumination. The sample 443, which is a chicken egg in this instance, is disposed on the sample holder 442 (e.g., egg holder) in a horizontal position.

The aperture 444 may be of any suitable size and shape (e.g., circle, rectangle, etc.) for passing illumination from the laser source 410 to the sample. For example, the aperture 444 may be a 1.4 mm×1.4 mm square aperture. The chamber 441 also has an opening 445 covered with a transparent plastic window to provide light scattered by the sample 443 to the second optical system 450 and onto the one or more light detectors 472. In some cases, the LSCI system 400 may also include an isolation chamber surrounding one or more components to prevent exposure to noises and stray light from the surrounding environment.

The first optical system 420 includes an optical shutter 422 placed after the laser source 410 to block the laser source 410 when the camera 470 is not recording or when speckle patterns are being recorded to determine a dark noise frame. A discussion of an example procedure for collecting speckle patterns for a dark noise frame is discussed with reference to FIG. 9. The optical shutter 422 may be digitally controlled according to one implementation. The first optical system 420 also has a pair of reflective mirrors including a first reflective mirror 424 and a second reflective mirror 426 in a face-to-face 90-degree configuration to direct the laser light in a first direction onto the sample 443 for side-illuminating the sample (e.g., chicken egg).

The scattered light exiting the sample 443 is collected from a second direction (e.g., from the top view in FIG. 4) by the second optical system 450, which provides collected light onto a camera 470. The first direction of the optical path of the laser light being provided onto the sample 443 is at an angle, e, (e.g., about 90 degrees) from the second direction of the optical path of light collected by the second optical system 350. In one aspect, the angle may be about 90 degrees. In another aspect, the angle may be between 45 degrees and 135 degrees. In another aspect, the angle may be about 180 degrees. Other angles may be used in other implementations.

The second optical system 450 includes a mirror 451 for changing the propagation orientation from vertical to horizontal. The second optical system 450 also includes an aperture 452 inserted next to the mirror 451. The aperture 452 may be adjustable to control the region-of-interest of the field-of-view and to filter out undesired stray light. The second optical system 450 also includes a tunable imaging lens 454 and an adjustable aperture 455. In one example, the tunable imaging lens 454 may have a 50 mm focal length (e.g., #86-574 lens sold by Edmund Optics). The adjustable aperture 455 is placed in the pupil plane of the tunable imaging lens 454 in front of the camera 470 to enable adjustment of the speckle size of the light field by adjusting the adjustable aperture 455. In one example, the speckle size is set to about two pixels per speckle. The second optical system 450 also includes an optical shutter 456 placed in front of the camera 470 that can be used to switch on/off raw speckle pattern (frame) acquisition. The optical shutter 456 may be digitally controlled according to one implementation.

The image detection system includes a camera 470 (e.g., camera with a 50 mm focal length lens such as the #86-574 lens sold by Edmund Optics) that can record emitted light from the sample 443. In one aspect, the camera 470 may be an RGB camera. In another aspect, the camera 470 may be a monochromatic camera. For example, the camera 470 may be the CS126CU RGB camera sold by Thorlabs with an exposure time ranging from 0.03 ms to 14.7 s. The CS126CU RGB camera has a camera resolution is 3000×4096 pixels and with a pixel pitch size of 3.45×3.45 μm. The CS126CU RGB camera has a quantum efficiency of approximately 20% at the laser source wavelength of 852 nm. In other implementations, a higher frame rate and/or higher quantum efficiency may be used. In one implementation, the camera 470 is set with an exposure time of 10 ms and at a speed (frame rate) of 21 frames-per-second.

In FIG. 4, LSCI system 400 also includes a computing system 480 with a display 486 including a plurality of graphical user interface (GUI) elements 487 for entering user settings for controlling imaging. The GUI elements 487 include an icon 481 for initiating a “live view mode.”

The GUI elements 487 also include elements that enable a user to enter settings for image acquisition on one or more samples. For example, GUI elements 487 include a first field 482 for entering a sample identifier, a second field 483 for entering a recording interval in minutes, and a third field 484 for entering the number of speckle patterns to capture during the recording interval. By entering the sample identifier, the LSCI system may control the placement of the corresponding sample by, for example, moving a sample holder 442 with an egg from the incubator 440 to position it for side illumination. The GUI elements 487 also includes a start button 488 for initiating image acquisition process. In one implementation, triggering the start button will initiate an automated process using the values entered into first field 482, second field 483, and third field 484, and that may not necessarily require additional input from a user. The display 486 also includes output including an image 485 (e.g., BFI map) of the blood vessels of sample 443 being imaged and a graph 489 of blood flow dynamics (e.g., BFI) over time (e.g., over 5 seconds).

The computing system 480 also includes one or more processors (e.g., processors and/or other circuitry 282 in FIG. 2) and a non-transitory computer readable medium (e.g., CRM 284 in FIG. 2) in electrical communication with the processor(s) or other circuitry. The computing system 480 is in electrical communication with optical shutter 456 and/or the one or more light detectors 472 to send control signals. Optionally, the computing system 480 may also be in electrical communication with the laser source 410 and optical shutter 422 to send control signals. The processor(s) or other circuitry of the computing system 480 and, additionally or alternatively, other external processor(s) can execute instructions stored on non-transitory computer readable media to perform operations of the LSCI system 400.

The internal computer readable medium of the computing system 480 can include a non-volatile memory for storing processor-executable code (or “instructions”) that is retrieved by one or more processors or other circuitry to perform various functions or operations described herein for carrying out various logic or other operations on the image data. The internal computer readable medium also can store raw image data, processed image data, and/or other data. In some implementations, the internal computer readable medium or a separate memory device can additionally or alternatively include a volatile memory array for temporarily storing code to be executed as well as image data to be processed, stored, or displayed. In some implementations, the computing system 480 itself can include volatile memory and in some instances also non-volatile memory.

LSCI system 400 may also include one or more communication interfaces in communication with various devices such as an external computing system, an external display, ana external memory device, etc. The computing system 480 may also include a network communication interface that can be used to receive information such as software or firmware updates or other data for download by the computing device.

In some implementations, the computing system 480 may be configured or configurable (e.g., by a user) to: (i) process raw speckle patterns and other raw data such as traces of blood vessel metrics as a function of time, (ii) output raw data, processed data, and/or other data to display 486, (iii) output raw image data as well as processed image data and other processed data to an external computing device, (iv) output raw image data as well as processed image data and other data for storage on an internal or external memory device, and/or (v) output raw image data as well as processed image data over a network communication interface for communication over an external network.

The described electrical communication between components of LSCI systems described herein may be able to provide power and/or communicate data. The electrical communication between components of the LSCI systems described herein may be in wired form and/or wireless form.

### III. LSCI Methods

When a laser light beam is directed onto a sample such as an avian egg, the light may experience random scattering events before exiting the sample. In various embodiments, an SCI system illuminates a sample from one direction (side) and collects light exiting the sample from another direction. The side incident light may be scattered in multiple directions by components within the sample, illuminating dynamic elements near the portion of the sample from which light is collected by the image detection system (e.g. blood vessels in CAM near side of eggshell where light is emitted and collected). The light collected by the image detection system captures a series of raw speckle patterns (frames) that are granular in appearance. Generally speaking, a “speckle pattern” refers to a pattern of bright and dark spots in an image frame resulting from scattering of incident coherent light (e.g., by elements of the egg such as the eggshell, blood cells, etc.) resulting from constructive and destructive interference. As dynamic components (such as blood cells) within the sample move, the speckle pattern dynamics change. The time it takes for one speckle pattern to change to a different speckle pattern is referred to as a “decorrelation time,” which can be correlated with blood metrics such as blood flow rate, heart rate, blood volume, and blood oxygenation. As the speckle field fluctuates, the speckle patterns recorded by the SCI system are smeared and washed out within the acquisition time. The dynamic components of a sample may be imaged and their rate of change of their components such as blood flow may be quantified from speckle contrast of (degree of blurring between) speckle patterns recorded by the image detection system. The speckle contrast is defined as the ratio of the standard deviation and the mean value of the pixel intensities in a recorded speckle patterns. To ensure the recorded speckle patterns represent the dynamics of the sample, the exposure time of the image detection system (e.g., camera) is generally set to be significantly longer than the decorrelation time. In some examples, a camera may use a frame rate in a range of about 30 frames per second-150 frames per second. In one example, the camera may have a frame rate of about 50 frames per second.

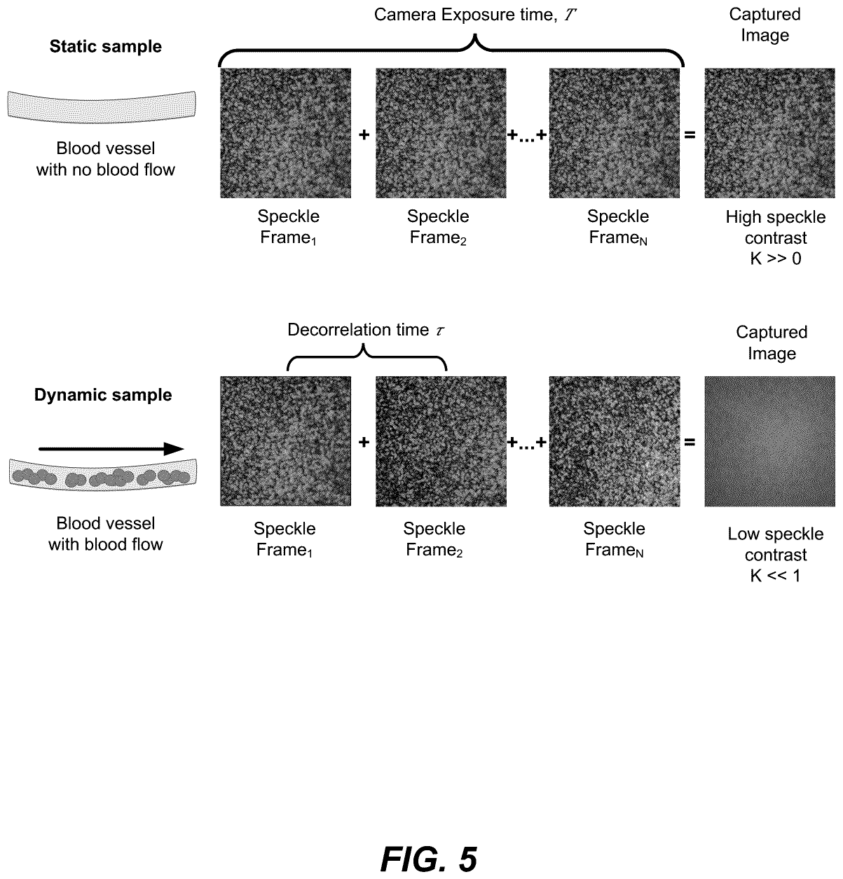

FIG. 5 is a schematic representation of speckle dynamics of living tissues, according to an embodiment. In a static sample such as a blood vessel with no blood flow, the N raw speckle patterns (frames) acquired during an exposure time (e.g., camera exposure time) do not change and the speckle contrast is greater than 0 (e.g., K>>0). In a dynamic/moving sample such as a blood vessel with blood flow, the N raw speckle patterns (frames) change and the captured image (e.g., sum of the N raw speckle patterns) is blurry. For the dynamic/moving sample, the speckle contrast is less than 1 (e.g., K<<1). To ensure the recorded speckle patterns represent the dynamics of the moving components, the exposure time, T, is set to be significantly longer than the decorrelation time, t.

In various implementations, the dynamic components (e.g., blood vessels) in a sample may be visualized and their rate of change determined based on speckle contrast of raw speckle patterns (frames) recorded by an LSCI system. Generally speaking, the speckle contrast of dynamic components in a sample is lower than the speckle contrast of static components in an image. For example, when imaging blood dynamics of blood cells moving through vessels in an egg, the moving blood cells in the vessels exhibit greater movements than the yolk and other background tissues (i.e. shorter decorrelation time). In this example, the speckle contrast values in the regions of the blood vessels are lower than the background tissues such that images based on speckle contrast show dynamic components with lower (e.g., brighter) values than the background tissues.

In certain embodiments, an LSCI method based on temporal speckle contrast is used, which can advantageously remove static scattering such as may be caused by cracks on an eggshell. Temporal speckle contrast may be determined for an imaging pixel (at row i and column j) in the temporal domain over a sequence of N speckle patterns (frames) captured during an exposure time as:

$${K}_{t} ( i , j ) = \frac{{σ}_{t}}{{μ}_{t}} = \frac{\sqrt{\frac{N}{N - 1} ( {〈 {\tilde{I}}^{2} ( i , j ; t ) 〉}_{t} - {( {〈 \tilde{I} ( i , j ; t ) 〉}_{t} )}^{2} )}}{{〈 \tilde{I} ( i , j ; t ) 〉}_{t}} \qquad ( Eqn . 1 )$$

In Eqn. 1 above, σₜ represents the temporal standard deviation of I, and μₜ represents the temporal mean of the N recorded speckle patterns, Ĩ(i, j; t) represents the dark noise subtracted intensity recorded at the image detection system (e.g., camera) at pixel row i and column j, t is the time at which the raw speckle pattern was recorded by the image detection system, and < >ₜ indicates temporal averaging over time occurring at pixel i,j, σₜ represents the standard deviation of I.

In certain embodiments, the LSCI method determines a time series of dynamic images (e.g., movie) by applying a sliding temporal window across the sequence of N speckle frames captured over the acquisition time. This time-varying temporal LSCI method (sometimes referred to herein as a “dynamic imaging method”) applies a sliding window with n raw speckle patterns (e.g., three raw speckle images, four raw speckle images, etc.) to the sequence of the N raw speckle patterns to determine adjacent sets of n raw speckle patterns. A temporal speckle contrast is determined for each set of n adjacent raw speckle frames at each imaging pixel (at row i and column j) in the temporal domain as:

$${K}_{t} ( i , j , {t}^{′} ) = \frac{\sqrt{\frac{n}{n - 1} ( {〈 {\tilde{I}}^{2} ( i , j ; t ) 〉}_{t} - {( {〈 \tilde{I} ( i , j ; t ) 〉}_{t} )}^{2} )}}{{〈 \tilde{I} ( i , j ; t ) 〉}_{t}} \qquad ( Eqn . 2 )$$

In Eqn. 2, t′ represents the indicator of the corresponding temporal sliding window. The temporal speckle contrast is calculated for t′=1 to N−n+1 where the sliding window has a stride (shifts) between adjacent sets of n frames across the sequence of N speckle frames. In one case, the stride is one frame where the sliding window shifts one frame between adjacent sets of n raw speckle patterns. For example, if a sliding window includes three (3) raw speckle frames (n=3) and the total number N of raw speckle frames acquired during an exposure time is one hundred (100 frames, the temporal speckle contrast is calculated for ninety eight (98=100−3+1) sets of speckle patterns to determine a sequence of ninety eight (98) speckle contrast images. In other implementations, the stride may be more than one (1). In one aspect, the sliding window includes at least three speckle frames.

The blood flow index (BFI) can be determined at an imaging pixel as:

$$BFI ( i , j , {t}^{′} ) = \frac{1}{{{K}_{t} ( i , j , {t}^{′} )}^{2}} \qquad ( Eqn . 3 )$$

The BFI provides relative blood flow information that correlates to the total volume of blood moving through a blood vessel during a time period. A change in BFI is indicative of one or both of a change in blood pressure and a change in diameter of the blood vessel. The heart rate can be determined by the periodicity of the pulsations of the blood flow dynamics. For example, the heart rate may be determined by taking a Fourier transform of time domain data of blood flow information (e.g., blood flow index) and determining the heart rate from the peak value in the frequency domain. As another example, the heart rate may be determined by calculating a time period between peaks in the time domain data of blood flow information.

### Dynamic Imaging

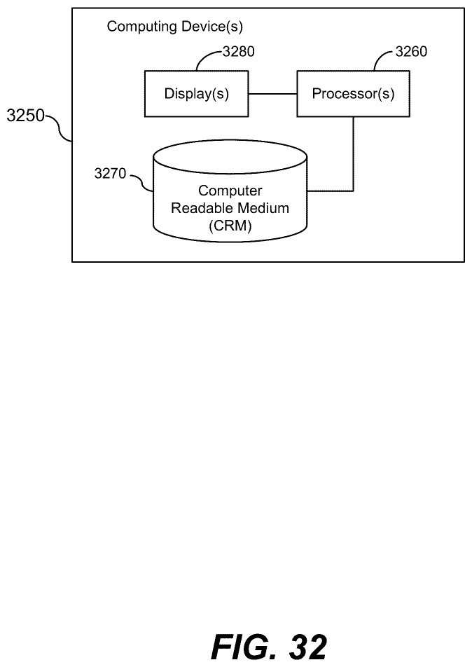

FIG. 6 is a flowchart of an example LSCI process 600 for generating a sequence of dynamic element images (e.g., BFI movie of blood vessels), according to some embodiments. Certain blocks of LSCI process 600 may be executed by one or more processors of one or more computing devices and other components of an LSCI system. An example of such a computing device is shown in and described below in connection with FIG. 32. In some implementations, blocks of LSCI process 600 may be executed in an order other than what is shown in FIG. 6. In some embodiments, one or more blocks of LSCI process 600 may be omitted, and/or two or more blocks may be executed substantially in parallel.

LSCI process 600 can begin at 602 by causing, using one or more laser sources, light to be emitted in a first direction onto a sample such as an avian egg. Examples of one or more laser sources are shown and described in connection with FIGS. 2, 3, and 4. In one example, a laser emitting infrared light may be used. Note that light may be emitted continuously, or may be pulsed. Scattering media within the sample may scatters the light. Light emitted from the sample is collected from a second direction at an angle from the first direction. The light is collected by an optical system, which propagates collected light to one or more light detectors for imaging.

At 604, LSCI process 600 may obtain, using the one or more light detectors, a sequence of speckle patterns capturing during an acquisition period. The speckle patterns are indicative of light scattered by scattering media within the sample. Examples of the one or more light detectors are shown and described in connection with FIGS. 2, 3, and 4. In some cases, the one or more light detectors may be part of an image detection system that includes a camera.

At 606, LSCI process 600 may optionally (denoted by dashed line) subtract a dark noise frame from each of the speckle patterns to determine speckle patterns with reduced or no dark noise. A dark noise frame may be estimated from speckle patterns acquired by the one or more light detectors when the one or more laser sources are blocked (e.g., blocked by an optical shutter) or turned off. An example of a procedure for estimating a dark noise frame is shown and described in connection with FIG. 9.

At 608, LSCI process 600 may apply a sliding window in the temporal domain in a stride across a sequence of N speckle patterns captured during the exposure time to determine overlapping sets of speckle patterns of n speckle patterns. The sliding window includes n adjacent speckle frames (e.g., two (2) frames, three (3) frames, four (4) frames, five (5) frames, etc.) and is typically applied in uniform strides (e.g., stride of 1 frame, stride of two frames, etc.) across the temporal domain. Where the stride is 1, N−n+1 sets of overlapping speckle patterns are determined. Within each sliding window, the n adjacent speckle frames can be used to reconstruct a temporal speckle contrast image using Eqn. 2. A speckle contrast image may be reconstructed from each of the overlapping sets of speckle images to determine a sequence of temporal speckle contrast images. A sequence of (N−n+1 where stride is 1) speckle contrast images may be determined from the corresponding overlapping sets of speckle patterns.

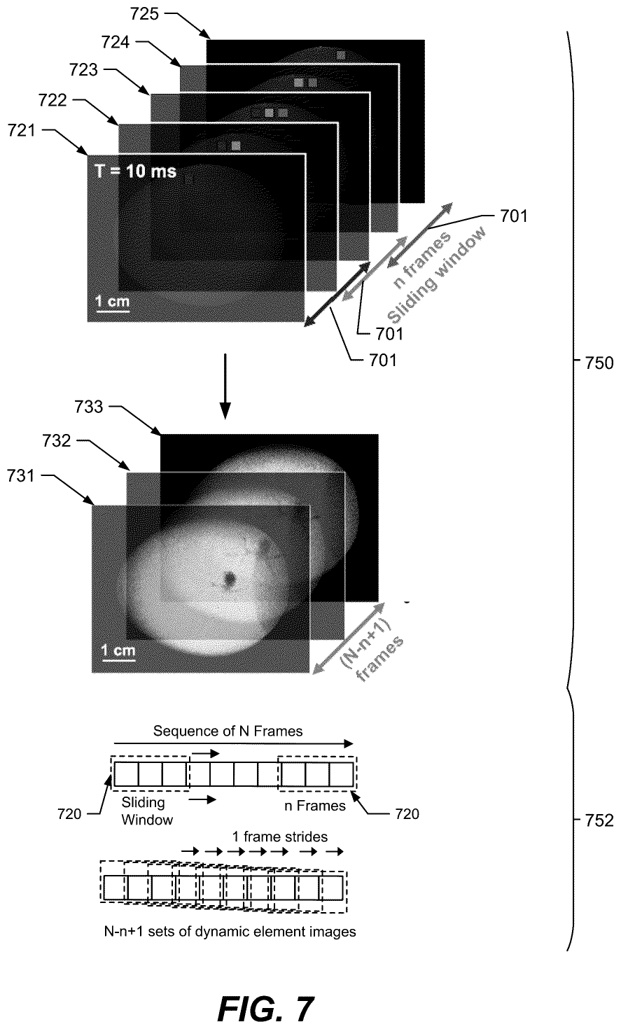

FIG. 7 is a schematic illustration depicting examples of a sliding window being applied in the temporal domain with a stride of 1 to sequences of speckle frames captured during an exposure time to determine overlapping sets of adjacent speckle patterns used to reconstruct a sequence of temporal speckle contrast images, according to implementations. In these examples, the sliding window 701, 720 at two different strides includes three (3) adjacent speckle frames (n=3). In the first example 750, a sequence of five speckle frames 702 (N=5) captured by one or more light detectors includes speckle frames 721, 722, 723, 724, and 725. The sliding window 701 includes three speckle frames (n=3). A sequence of three (N−n+1) temporal speckle contrast images 731, 732, and 733 may be reconstructed from the corresponding three adjacent overlapping sets of three speckle frames (i.e. first set 712, 722, 723, second set 722, 723, and 724, and third set 723, 724, and 725). The sliding window shifts by one stride (one frame) across the sequence of N frames 702. In the second example 752, a sequence of ten (10) speckle frames 702 (N=10) is captured by one or more or more light detectors and the sliding window includes three frames (n=3). A sequence of eight (N−n+1) temporal speckle contrast images may be reconstructed from the eight adjacent overlapping sets of three speckle frames in the sliding window shifted by one stride (one frame) across the sequence of ten speckle frames.

According to various embodiments, a sequence of temporal speckle contrast images is reconstructed from adjacent overlapping sets of speckle frames defined by a sliding window shifted by a stride across a sequence of raw speckle images captured during an exposure time. In some cases, the sets of overlapping speckle frames may overlap by at least one frame. In one aspect, the sets of overlapping speckle frames overlap by one frame. In another aspect, the sets of overlapping speckle frames overlap by two frames. In another aspect, the sets of overlapping speckle frames overlap by three frames.

At optional 610, LSCI process 600 may apply a denoising filter procedure to reduce speckle noise in the sequence of temporal speckle contrast images. An example of a denoising filter procedure that can be used is an anisotropic diffusion filter (ADF) which can improve image details while enabling image smoothing. Applying a denoising filter procedure may advantageously improve signal-to-noise ratio (SNR) for individual pixels which enables better contrast detection of blood vessels.

At 612, LSCI process 600 may convert the sequence of temporal speckle contrast images to a sequence of dynamic element images (e.g., extraembryonic blood vessel images). For example, to quantity blood flow, the sequence of speckle contrast images may be converted to a sequence of blood flow index (BFI) images (e.g., BFI movie) using Eqn. 3. The sequence of blood flow index (BFI) images may be used to visualize extraembryonic blood vessel dynamics in live view mode, for example.

At optional 614, LSCI process 600 may apply a Fourier high-pass filter to the sequence of dynamic element images to determine a sequence of filtered dynamic element images (e.g., filtered movie). For example, for each pixel, the signal in the temporal domain may be Fourier transformed, then multiplied by the high-pass, and then inverse Fourier transformed back, to determine the sequence of filtered dynamic element images. The Fourier high-pass filter may be used to extract higher frequency element dynamics such as the heartbeat of the embryo from the lower frequency background variations.

In one embodiment, the LSCI process 600 may also take the average of the filtered dynamic element images in the temporal domain to determine a temporally-averaged dynamic element image, which may advantageously remove imaging artifacts. In one aspect, the temporally-averaged dynamic element image of the extraembryonic blood vessels of an avian egg may be used to predict the developmental stage of the embryo.

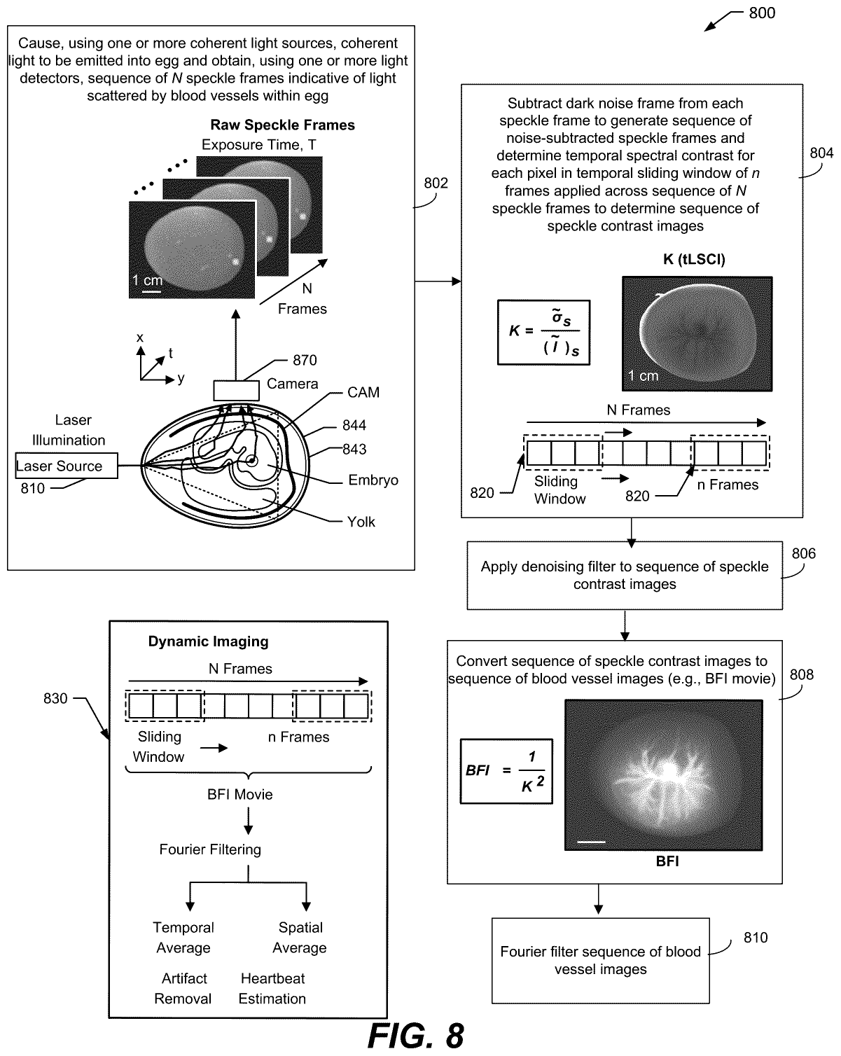

FIG. 8 is a schematic diagram of an example LSCI process 800 for generating a sequence of extraembryonic blood vessel images of an avian egg 843 with an eggshell 844, according to some embodiments. For example, the sequence of extraembryonic blood vessel images may be a BFI movie for visualizing blood dynamics of the extraembryonic blood vessels of the avian egg 843. In certain embodiments, an eggshell (e.g. eggshell 844 in FIG. 8) of an avian egg being examined is an approximately 300 μm thick calcite eggshell.

Certain blocks of LSCI process 800 in FIG. 8 may be executed by one or more processors of one or more computing devices and other components of an LSCI system. An example of such a computing device is shown in and described below in connection with FIG. 32. In some implementations, blocks of LSCI process 800 may be executed in an order other than what is shown in FIG. 8. In some embodiments, one or more blocks of LSCI process 800 may be omitted, and/or two or more blocks may be executed substantially in parallel.

LSCI process 800 can begin at 802 by causing, using a laser source 810, a laser beam to be emitted in a first direction onto the avian egg 843. The laser source 810 may be a near infrared laser source. The light will experience multiple scattering events before exiting the avian egg 843. At 802, the LSCI process may also obtain, using a camera 870 with one or more light detectors, a sequence of speckle patterns capturing during an exposure time, T. Examples of the one or more light detectors are shown and described in connection with FIGS. 2, 3, and 4.

At 804, LSCI process 800 may subtract a dark noise frame from each of the speckle patterns to determine speckle patterns with reduced or no dark noise. A dark noise frame are estimated from speckle patterns captured by the one or more light detectors when the one or more laser sources are blocked or turned off. An example of a procedure for estimated a dark noise frame is shown and described in connection with FIG. 9. At 804, the LSCI process 800 may also apply a sliding window in the temporal domain in a stride across the sequence of N speckle patterns to determine sets of n speckle patterns that are used to determine a sequence of speckle contrast images. The sliding window typically includes n adjacent speckle frames (e.g., two (2) frames, three (3) frames, four (4) frames, etc.) and is applied in uniform strides (e.g., stride of 1 frame, stride of two frames, etc.) across the temporal domain. The sliding window is applied in a stride across a sequence of N speckle patterns to determine sets of n speckle patterns. The sets of speckle patterns within each window location are typically overlapping by at least one speckle frame. Within each sliding window location, the n speckle frames can be used to reconstruct a speckle contrast image using Eqn. 2. A sequence of speckle contrast images can be constructed from corresponding overlapping sets of speckle images using Eqn. 2.

At 806, LSCI process 800 may apply a denoising filter procedure to reduce speckle noise in the sequence of temporal speckle contrast images. An example of a denoising filter procedure that can be used is an anisotropic diffusion filter (ADF) which can determine image details while enabling image smoothing.

At 808, LSCI process 800 may convert the sequence of temporal speckle contrast images to a sequence of blood flow images using Eqn. 3. The sequence of blood flow index (BFI) images (also sometimes referred to herein as “a BFI movie”) may be used to visualize blood vessel dynamics in a live view mode, for example.

At 810, LSCI process 800 may apply a Fourier high-pass filter to the sequence of blood flow index (BFI) images. For example, for each pixel in the BFI movie, the signal in the temporal domain may be Fourier transformed, multiplied by the high-pass filter, and then inverse Fourier transformed back to determine a filtered BFI movie. The Fourier high-pass filter may extract the higher frequency blood flow dynamic such as the heartbeat of the embryo from the lower frequency background variations. In one aspect, a Fourier high-pass filter may have a cut-off frequency that is smaller than the heartbeat frequency of the embryo.

In one implementation, the camera 870 may record a first sequence of N=200 speckle frames when the laser was on and a second sequence of N=200 speckle frames when the laser was off/blocked with the same exposure time of 10 ms. The recorded noise frames (no laser exposure) from the second sequence may be averaged to estimate a noise frame and the noise frame subtracted from each speckle frame of the first sequence to determine a sequence of noise-subtracted speckle frames. Using Eqn. 2, temporal speckle contrast values may be calculated for each camera pixel in the temporal domain over the N sequence of noise-subtracted speckle frames to reconstruct speckle contrast images. As blood cells move within the blood vessels, the movement may generate multiple speckle realizations which may lead to a smeared and washed out image of the speckles recorded by the camera over the exposure time and a corresponding low speckle contrast value. Applying a sliding window and using temporal speckle contrast values in the temporal domain can advantageously preserve image details while enabling smoothing. In one aspect, a sliding window of at least three frames (n≥3) is used for the reconstructions. In this implementation, a denoising procedure may also be applied to improve signal-to-noise ratio (SNR) for individual pixels which allows for improved speckle contrast detection of blood vessels. The reciprocal of the squared speckle contrast, K², may be converted to blood flow index values to qualify the blood flow to determine a sequence of BFI images. A Fourier high-pass filter may be applied to the BFI images to extract blood flow dynamics (e.g., heartbeat of the embryo) from the low frequency background variations.

### Dark Noise Frame Estimation

FIG. 9 is a flowchart depicting an example LSCI process 900 for estimated a dark noise frame from a sequence of raw speckle patterns, according to an embodiment. Blocks of LSCI process 900 may be executed by one or more processors of one or more computing devices. An example of such a computing device is shown in and described below in connection with FIG. 32. In some implementations, blocks of LSCI process 900 may be executed in an order other than what is shown in FIG. 9. In some embodiments, one or more blocks of LSCI process 900 may be omitted, and/or two or more blocks may be executed substantially in parallel. This process may occur as part of a calibration procedure for the LSCI system.

At 902, LSCI process 900 may obtain, using the one or more light detectors, a sequence of speckle patterns capturing during an acquisition period. The time duration of this acquisition period may be the same time duration of the acquisition period used to acquire the speckle patterns at 604 of process 600. During this acquisition period, the one or more laser sources are blocked (e.g., blocked by optical shutter 322 in FIG. 3) or turned off such that there is no laser exposure.

At 904, LSCI process 900 may take an average of the sequence of speckle patterns captured when the one or more laser sources are blocked/off to estimate a dark noise frame. For example, the dark noise frame can be estimated by averaging N=200 raw speckle frames acquired while the one or more laser sources are blocked/off.

### Heart Rate

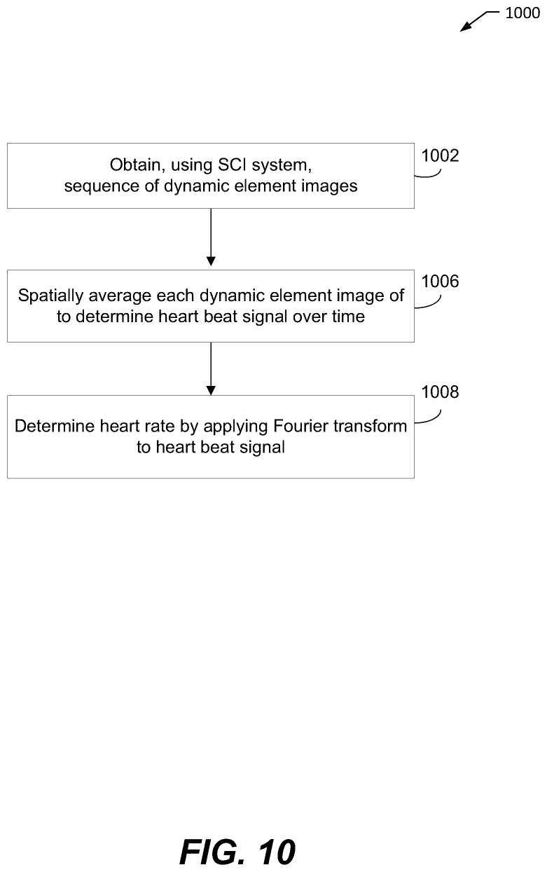

FIG. 10 is a flowchart depicting an example LSCI process 1000 for determining heart rate, according to an embodiment. Blocks of LSCI process 1000 may be executed by one or more processors of one or more computing devices. An example of such a computing device is shown in and described below in connection with FIG. 32. In some implementations, blocks of LSCI process 1000 may be executed in an order other than what is shown in FIG. 10. In some embodiments, one or more blocks of LSCI process 1000 may be omitted, and/or two or more blocks may be executed substantially in parallel.

At 1002, LSCI process 1000 may obtain, using one or more components of an LSCI system, a sequence of dynamic element images. For example, a sequence of dynamic element images may be determined using certain operations of LSCI process 600 shown and described in connection with FIG. 6 or certain operations of LSCI process 800 shown and described in connection with FIG. 8.

At 1006, LSCI process 1000 may determine a heartbeat signal by spatially averaging each of the dynamic element images over time. The sequence of dynamic element images may be determined using, for example, certain operations of LSCI process 600 shown and described in connection with FIG. 6 or certain operations of LSCI process 800 shown and described in connection with FIG. 8. In some cases, a Fourier high-pass filtered sequence of dynamic element images (e.g., Fourier high-pass filtered BFI movie) at 614 of process 600 can be used.

For example, the heartbeat signal, S_{HB}, can be determined by spatially averaging a Fourier filtered BFI movie as follows:

$${S}_{HB} ( {t}^{′} ) = \frac{1}{{N}_{x} \cdot {N}_{y}} \cdot {\sum }_{i , j} {BFI}_{f} ( i , j , t ) \qquad ( Eqn . 4 )$$

In Eqn. 4, Nₓ and N_{y} represent the pixel number identifiers of the camera region of interest in the horizontal and vertical directions respectively. The heartbeat signal, S_{HB}, is calculated for i=1 to Nx, j=1 to Ny, and t′=1 to N−n+1.

At 1008, LSCI process 1000 may determine a heart rate by applying a Fourier transform to the heartbeat signal.

### Developmental Stage Classification

Accurate staging of avian embryos is important for early developmental biology studies, particularly in monitoring the development of organs such as the heart, limbs, retina, and other vital structures. Conventionally, staging involved opening the eggshell and membrane, followed by a meticulous examination of the embryo conducted by experienced biologists. Certain LSCI techniques described herein can advantageously image internal elements within the eggshells and can stage eggs non-invasively, which enables longitudinal studies with the same eggs.

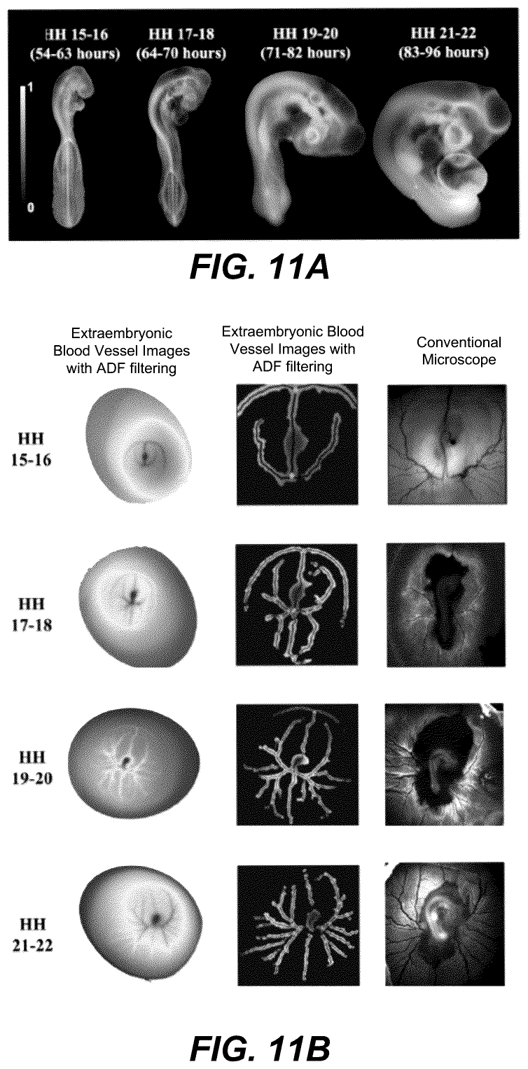

The development of an avian embryo is a continuous process, which is commonly studied by grouping adjacent Hamburger-Hamilton (HH) stages of the forty six (46) HH developmental stages. For example, groupings may include a series including a first group with stages HH15-16, a second group with stages HH17-18, a third group with stages HH19-20, and a fourth group with stages HH21-22. A stage period from stages HH15 to HH22 marks a fast-growing period in blood vessel expansion. FIG. 11A depicts example images of the four Hamburger-Hamilton (HH) developmental stages HH15-16, HH17-18, HH19-20, and HH21-22 captured with a wide-field microscope. FIG. 11B depicts example extraembryonic blood vessel images determined with an LSCI method without ADP filtering, with an LSCI method with ADP filtering, and with a conventional wide-field microscope for comparison.

Disclosed herein are LSCI techniques for using blood vessel metrics to classify the developmental stage of an avian embryo. In particular, these LSCI techniques may utilize blood vessel metrics including extracted features such as one or more of an extraembryonic blood vessel length, a number of branches in the extraembryonic blood vessel network, an extraembryonic blood vessel area, and a standard deviation of blood flow index (BFI), to classify the developmental stage. The blood vessel metrics may be obtained based on speckle patterns acquired during an exposure time of an image detection system (e.g., camera) of various LSCI systems described herein. In various embodiments, a trained machine learning model is provided with blood vessel data including one or more extracted features, one or more raw speckle patterns, and/or one or more extraembryonic blood vessel images based on temporal speckle contrast, to classify the developmental stage. In one embodiment, a trained machine learning model is provided with blood vessel data with extracted features including one or more of an extraembryonic blood vessel length, a number of branches in extraembryonic blood vessel network, an extraembryonic blood vessel area, and a standard deviation of blood flow index.

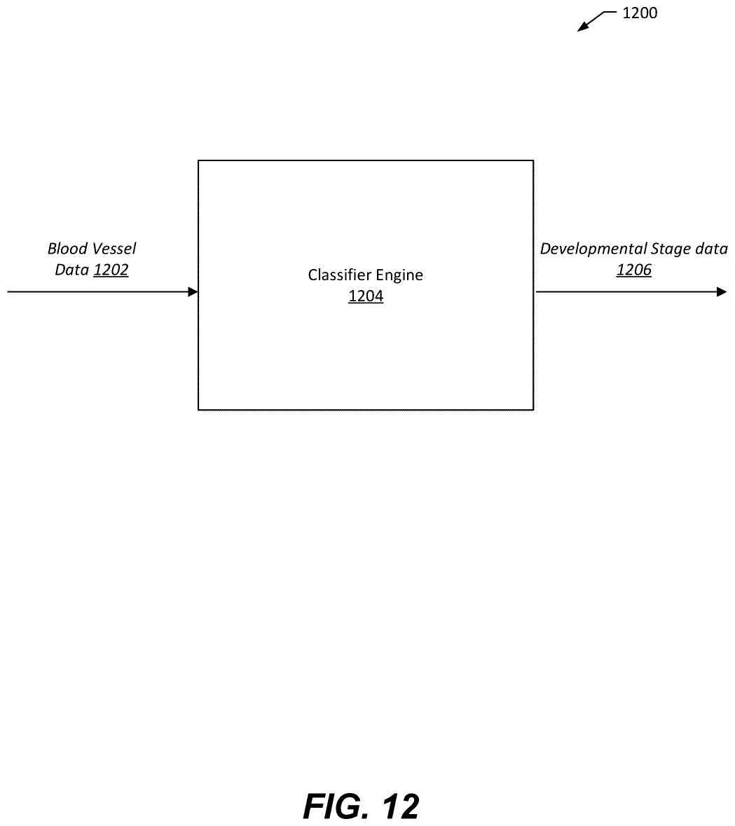

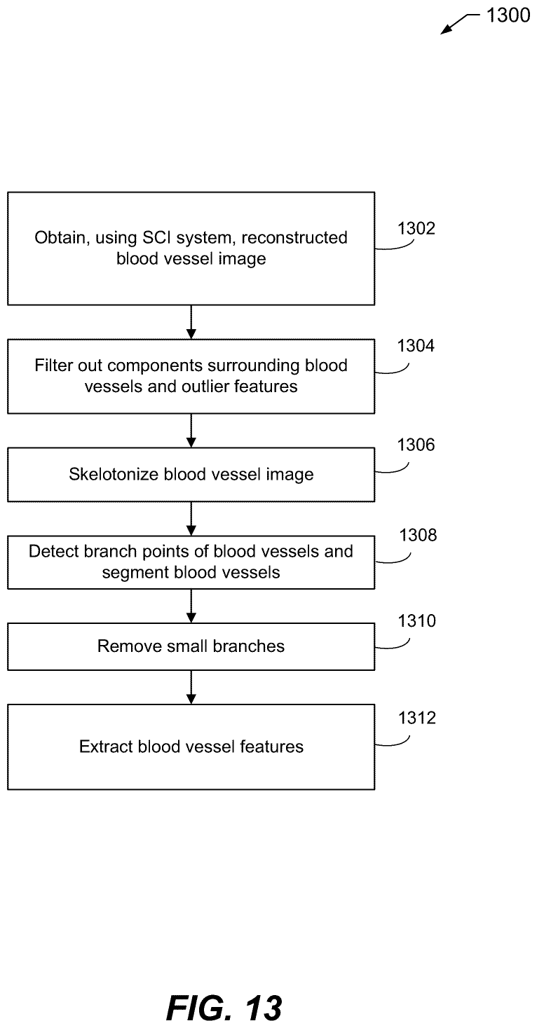

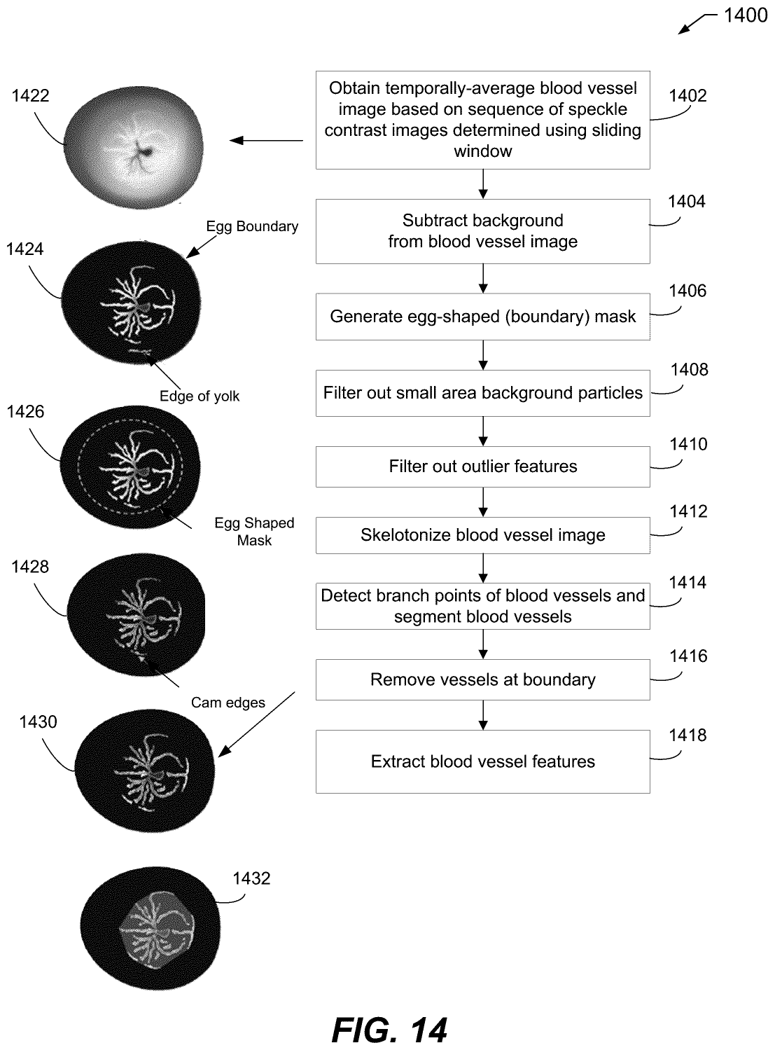

FIG. 12 is a block diagram of an example system 1200 for classifying a developmental stage of an avian embryo in accordance with some embodiments. As illustrated, the system 1200 includes a classifier engine 1204. In some implementations, classifier engine 1204 may include a trained machine learning model configured to take, as input, blood vessel data 1202, and generate, as an output, developmental stage data 1206. The trained machine learning model may be a perceptron, a random forest, decision tree, light gradient-boosting machine, K-nearest neighbors, logistic regression, Naïve Bayes, support vector classifier (SVC), a deep neural network (DNN) such as a convolution neural network, or any other suitable architecture. In certain implementations in which blood vessel data 1202 includes extracted blood vessel features, the trained machine learning model may be an SVC. Such an SVC may be able to identify features in the image data not observable or identifiable by a human that are useful for classifying the developmental stage of the avian embryo being imaged non-invasively. In implementations in which blood vessel data 1202 includes extracted blood vessel features (as described in more detail in connection with FIGS. 13 and 14) the trained machine learning model may be a perceptron, a random forest, or other type of architecture configured to take extracted features as input and classifying developmental stage of an avian egg.

The developmental stage data 1206 may be a number on a discrete scale (e.g., an integer between 1 and 46 corresponding to the forty-six (46) HH developmental stages, an integer in a range corresponding to different HH stage periods, etc.), a number on a continuous scale, or the like. In some cases, the developmental stage data 1206 may represent a likelihood that, given the blood vessel data 1202, the sample is in a developmental stage period (e.g., a stage period including stages HH15-16, stages HH17-18, stages HH19-20, or stages HH21-22).

It should be noted that classifier engine 1204 may be implemented by one or more computing devices or one or more processors. For example, such a computing device and/or processor may be configured to analyze data from one or more light detectors, generate blood vessel images, extract blood vessel features from blood vessel images, and provide data representative of the blood vessel metrics to a trained machine learning model to generate developmental stage data 1206.

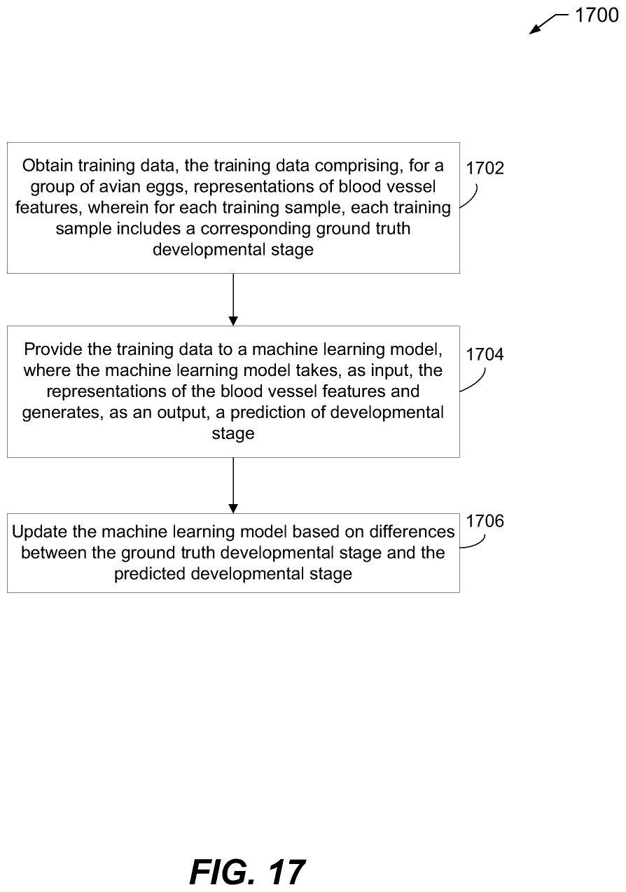

In some implementations, raw speckle patterns or blood vessel images may be provided to a trained machine learning model configured to output a developmental stage. Such a machine learning model that accepts blood vessel images may be a DNN or other suitable architecture. Alternatively, in some embodiments, one or more extracted blood vessel features may be provided to a trained machine learning model configured to output a developmental stage. Such a machine learning model that accepts extracted features may be a support vector classifier (SVC), gradient-boosting machine, K-nearest neighbors, logistic regression, Naïve Bayes, or any other suitable architecture. The extracted features may then be provided to a trained machine learning model, which in turn may classify a developmental stage of the egg. The extracted features may be considered “a representation of the one or more blood vessel metrics.” Note that techniques for classifying a developmental stage using a machine learning model are shown in and described below in connection with FIG. 16, and techniques for training such a model are shown in and described below in connection with FIG. 17.

In some implementations, extracted features may include information captured during an exposure time of an image detection system of certain SCI systems described herein such as LSCI systems 200, 300, and 400 shown in and described in connection with FIGS. 2, 3, and 4. Extracted features may include blood vessel features such as blood vessel length, number of branches, blood vessel area, number of blood vessels, standard deviation of the BFI values, heart morphology, heart rate, and blood flow cardiac cycle waveform features.

In some implementations, extracted features may include a normalized standard deviation of blood flow index (BFI) in a sequence of speckle patterns. The standard deviation of BFI may be calculated by taking the standard deviation of the BFI values from all pixels. The BFI map can be calculated from the speckle contrast calculation (Eqns. 1-3)

Note that extracted features may be extracted and/or determined autonomously (e.g., without user input) upon collection of the one or more blood vessel images. The extracted features may then be provided to a trained machine learning model configured to classify the developmental stage. Note that any suitable number or combination of extracted features may be utilized.

Alternatively, in some embodiments, rather than utilizing a trained machine learning model, a developmental stage may be classified based on extracted features, e.g., by comparing an extracted feature to one or more predetermined thresholds. By way of example, developmental stage may be classified by determining whether a value of a particular extracted feature (e.g., blood vessel area, number of branches, blood vessel length, etc.) exceed a predetermined threshold.

FIG. 13 is a flowchart of an example LSCI process 1300 for extracting features from a blood vessel image, according to embodiments. In some cases, the blood vessel image may be a temporally-averaged blood vessel image determined by taking the average of a sequence of blood vessel images in the temporal domain. For example, the blood vessel image may be the average of sequence of blood vessel images determined using LSCI process 600 or LSCI process 800 shown in and described in connection with FIGS. 6 and 8 respectively. Certain blocks of LSCI process 1300 may be executed by one or more processors of one or more computing devices and other components of an LSCI system. An example of such a computing device is shown in and described below in connection with FIG. 32. In some implementations, blocks of LSCI process 1300 may be executed in an order other than what is shown in FIG. 13. In some embodiments, one or more blocks of LSCI process 1300 may be omitted, and/or two or more blocks may be executed substantially in parallel.

LSCI process 1300 can begin at 1302 by obtaining, using an LSCI system, a reconstructed blood vessel image based on a sequence of temporal speckle contrast images. In one example, the reconstructed blood vessel image may be a temporally-averaged blood vessel image determined by taking the average of a sequence of blood vessel images that are determined based on temporal speckle contrast. For example, the sequence of blood vessel images may be reconstructed from speckle patterns using an LSCI method described herein such as LSCI process 600 or LSCI process 800 shown in and described in connection with FIGS. 6 and 8 respectively. In one implementation, the reconstructed blood vessel image is obtained by taking the average of a sequence of blood vessel images determined from operations 6022, 604, 606, 608, 610, 612, and 614 shown in and described in connection with FIG. 6.

LSCI process 1300 can begin at 1302 by obtaining, using an LSCI system, a reconstructed blood vessel image based on a sequence of temporal speckle contrast images. In one example, the reconstructed blood vessel image may be a temporally-averaged blood vessel image determined by taking the average of a sequence of blood vessel images that are determined based on temporal speckle contrast. For example, the sequence of blood vessel images may be reconstructed from speckle patterns using an LSCI method described herein such as LSCI process 600 or LSCI process 800 shown in and described in connection with FIGS. 6 and 8 respectively. In one implementation, the reconstructed blood vessel image is obtained by taking the average of a sequence of blood vessel images determined from operations 6022, 604, 606, 608, 610, 612, and 614 shown in and described in connection with FIG. 6.

At 1304, LSCI process 1300 may filter out components surrounding the blood vessels such as the background components and outlier features. For example, a procedure such as a rolling ball-based background subtraction procedure may be applied to substantially subtract out the background of the blood vessels from the blood vessel image. In addition or alternatively, a threshold filter may be applied to remove small background particles with smaller area occupation. For example, the blood vessel image may be fed into a thresholding procedure (e.g., Li-method) to generate a binary mask.

The binary mask may have a shrinkage portion (e.g., 25%, 30%, etc.) of the egg boundary. An area threshold may be applied to the blood vessel image to filter out the small background particles with smaller area occupation. The blood vessel image may be multiplied by the egg-shaped mask (e.g., shown in example image 1426 in FIG. 14) to filter out the outlier portions such as the egg boundary outline and edges of the egg yolk as shown in example image 1424 in FIG. 14.

At 1306, SCI process 1300 may skeletonize the blood vessel image. For that, contrast thresholds or gradient mapping filters can be used to skeletonize the blood vessel image. In one case, spurious edges of the skeletonized image are also removed such as the CAM outside ring.

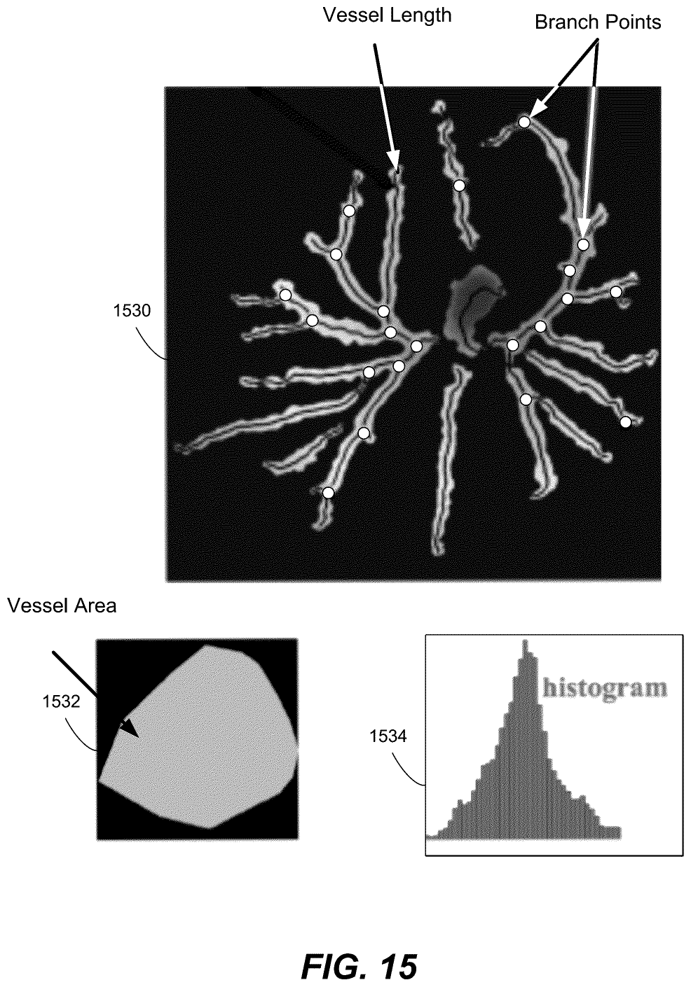

At 1308, LSCI process 1300 may detect one or more branch points in the blood vessels of the blood vessel image and segment the blood vessel image into blood vessel branches. An example of branch points is shown in and described in connection with FIG. 15.

At 1310, SCI process 1300 may apply a vessel length filter to remove one or more small branches. In one example, one or more blood vessels with high overlapping proportion with the convex hull boundary of the blood vessel image are removed.

At 1312, SCI process 1300 may extract one or more blood vessel features in the blood vessel image. Some examples of blood vessel features that may be extracted include vessel length of each of the branches, maximum vessel length of all the branches, total blood vessel area for all branches, maximum vessel area, maximum vessel length (maximum length) of all the branches, number of branches, and/or a standard deviation of the blood flow index distribution in the blood vessel image. Other features can include blood vessels, standard deviation of the BFI values, heart morphology, heart rate, and blood flow cardiac cycle waveform features.

In some cases, one or more of the blood vessel features may be quantified as numbers. For example, a maximum vessel length of all the branches may be calculated as the total length of vessel skeleton excluding the outer ring. As another example, the number of branches may be calculated as the number of branch points plus the number of isolated blood vessels which are not connected to other vessel branches. As another example, the vessel area for all the branches may be calculated as the total number of pixel counts (pixel areas) within the convex hull of all the blood vessels. The normalized standard deviation of BFI may be calculated based on its histogram, which is its standard deviation divided by its mean. An example of a histogram 1534 is shown in FIG. 15.

FIG. 14 is a flowchart of an example LSCI process 1400 for extracting features from a blood vessel image. In some cases, the blood vessel image may be a temporally-averaged blood vessel image determined by taking the average of a sequence of blood vessel images in the temporal domain. For example, the blood vessel image may be the average of sequence of blood vessel images determined using LSCI process 600 or LSCI process 800 shown in and described in connection with FIGS. 6 and 8 respectively. Certain blocks of LSCI process 1400 may be executed by one or more processors of one or more computing devices and other components of an LSCI system. An example of such a computing device is shown in and described below in connection with FIG. 32. In some implementations, blocks of LSCI process 1400 may be executed in an order other than what is shown in FIG. 14. In some embodiments, one or more blocks of LSCI process 1400 may be omitted, and/or two or more blocks may be executed substantially in parallel.

LSCI process 1400 can begin at 1402 by obtaining, using an LSCI system, a reconstructed blood vessel image based on a sequence of temporal speckle contrast images. In one example, the reconstructed blood vessel image (e.g., reconstructed image 1422 in FIG. 14) may be a temporally-averaged blood vessel image determined by taking the average of a sequence of blood vessel images that are determined based on temporal speckle contrast. For example, the sequence of blood vessel images may be reconstructed from speckle patterns using an LSCI method described herein such as LSCI process 600 or LSCI process 800 shown in and described in connection with FIGS. 6 and 8 respectively. In one implementation, the reconstructed blood vessel image is obtained by taking the average of a sequence of blood vessel images determined from operations 602, 604, 606, 608, 610, 612, and 614 shown in and described in connection with FIG. 6.

At 1404, LSCI process 1400 may substantially subtract out the background from the reconstructed blood vessel image. For example, a procedure such as a rolling ball-based background subtraction procedure may be used to substantially subtract out the background from the blood vessel image.

At 1406, LSCI process 1400 may generate an egg-shaped boundary mask such as shown in image 1426 in FIG. 14. For example, the blood vessel image may be fed into an auto-threshold procedure such as an Li-method to generate the egg-shaped boundary mask.

At 1408, LSCI process 1400 may filter out small area background particles from the blood vessel image. For example, a threshold filter may be applied to remove small background particles with smaller area occupation. For example, a Matlab function bwareaopen(BW,P) can be used, which is defined as “removes all connected components (objects) that have fewer than P pixels from an binary image BW, producing another binary image, BW2”. This operation is sometimes referred to as area opening.

At 1410, LSCI process 1400 may filter out outlier features from the blood vessel image by multiplying the blood vessel image with a binary mask which holds a shrinkage proportion (e.g., 25%) of the egg boundary mask. This step is to remove the background noise and outlier features that are located outside the egg boundaries and are not related to the blood vessels or cardiac blood flow of the embryo.

At 1412, LSCI process 1400 may skeletonize the blood vessel image. The skeletonize blood vessel image provides a straightforward support for measuring the number of branches or the location of the blood vessels.

At 1414, LSCI process 1400 may detect one or more branch points in the blood vessels of the blood vessel image and segment the blood vessel image into blood vessel branches. An example of branch points is shown in FIG. 14.

At 1416, LSCI process 1400 may apply a vessel length filter to remove one or more small branches. In one example, one or more blood vessels with high overlapping proportion with the convex hull boundary of the blood vessel image are removed. An example of a blood vessel image 1430 with small branches removed is shown in FIG. 14.

At 1418, LSCI process 1400 may extract one or more blood vessel features in the blood vessel image. Some examples of blood vessel features that may be extracted include vessel length of each of the branches, maximum vessel length of all the branches, total blood vessel area for all branches, maximum vessel area, maximum vessel length (maximum length) of all the branches, number of branches, and/or a standard deviation of the blood flow index distribution in the blood vessel image.

In some cases, one or more of the blood vessel features area may be quantified as numbers. For example, the maximum vessel length of all the branches may be calculated as the total length of vessel skeleton excluding the outer ring. As another example, the number of branches may be calculated as the number of branch points plus the number of isolated regions. As another example, the vessel area for all the branches may be calculated as the total number of pixel counts (pixel areas) within the convex hull of all the blood vessels. The normalized standard deviation of BFI may be calculated based on its histogram, which is its standard deviation divided by its mean. An example of a histogram 1534 is shown in FIG. 15.

FIG. 15 illustrates images depicting examples of features that may be extracted using certain LSCI processes described herein such as LSCI process 1300 and LSCI process 1400 shown in and described in connection with FIGS. 13 and 14. For example, a vessel length of one branch may be determined from blood vessel image 1530 by calculating the length from the branchpoint outward and the number of branches calculated as the number of branch points plus the number of isolated regions in the blood vessel image 1530. As another example, an estimated vessel area may be calculated as the total number of pixel counts (pixel areas) within the convex hull of all the blood vessels as shown in image 1530. As another example, the normalized standard deviation of BFI may be calculated based on its histogram 1534, which is its standard deviation divided by its mean. An example of segmentation, skeleton, branch points, coverage area and BFI spatial distribution overlayed together is shown in image 1432 in FIG. 14.

FIG. 16 is a flowchart of an example LSCI process 1600 for determining a developmental stage using blood vessel data in accordance with some embodiments. Blocks of process 1600 may be executed by one or more processors of one or more computing devices. An example of such a computing device is shown in and described below in connection with FIG. 32. In some implementations, blocks of LSCI process 1600 may be executed in an order other than what is shown in FIG. 16. In some embodiments, one or more blocks of process 1600 may be omitted, and/or two or more blocks may be executed substantially in parallel.

LSCI process 1600 can begin at 1602 by causing, using one or more laser sources, light to be emitted in a first direction onto an avian egg. Examples of one or more laser sources are shown and described in connection with FIGS. 2, 3, and 4. In one example, a laser emitting infrared light may be used. Note that light may be emitted continuously, or may be pulsed. Scattering media within the eggshell may scatters the light. Light emitted from the egg is collected from a second direction at an angle from the first direction. The light is collected by an optical system, which propagates collected light to one or more light detectors for imaging.

At 1604, LSCI process 1600 may obtain, using the one or more light detectors, a sequence of speckle patterns capturing during an acquisition period. Examples of the one or more light detectors are shown and described in connection with FIGS. 2, 3, and 4. In some cases, the one or more light detectors may be part of an image detection system that includes a camera.

At 1606, LSCI process 1600 may reconstruct a blood vessel image based on the sequence of speckle patterns capturing during an acquisition period. To reconstruct a sequence of temporal speckle contrast images, the LSCI process 600 may apply a sliding window in the temporal domain in a stride across a sequence of N speckle patterns captured during the exposure time to determine sets of n speckle patterns that are used to determine a sequence of (N−n+1) speckle contrast images. Within each sliding window, the n adjacent speckle frames can be used to reconstruct a temporal speckle contrast image using Eqn. 2. In some cases, the reconstructed speckle contrast images are averaged to determine a temporally-averaged speckle contrast image. The speckle contrast values at each imaging pixel may be converted to BFI values using Eqn. 3 to reconstruct the blood vessel image, which is a temporally-averaged BFI distribution map.

At 1608, LSCI process 1600 may extract one or more blood vessel features from the reconstructed blood vessel image. Some examples of blood vessel features include vessel length of each of the branches, maximum vessel length of all the branches, total blood vessel area for all branches, maximum vessel area, maximum vessel length (maximum length) of all the branches, number of branches, and/or a standard deviation of the blood flow index distribution in the blood vessel image. These features may be extracted using LSCI processes described above such as LSCI process 1300 or LSCI process 1400 shown and described in connection with FIGS. 13 and 14. In one example, a maximum vessel length of all the branches may be calculated as the total length of vessel skeleton excluding the outer ring. As another example, the number of branches may be calculated as the number of branch points plus the number of isolated regions. As another example, the vessel area for all the branches may be calculated as the total number of pixel counts (pixel areas) within the convex hull of all the blood vessels. The normalized standard deviation of BFI may be calculated based on its histogram, which is its standard deviation divided by its mean. An example of a histogram 1534 is shown in FIG. 15.

At 1610, process 1600 may provide a representation of the one or more blood vessel features as input to a trained machine learning model. Note that the representation of the one or more blood vessel features may include values of blood vessel features taken as an average or as a function of time from the blood vessel image.

At 1610, process 1600 may determine an indication of a developmental stage of the avian egg based on an output of the trained machine learning model. In one example, the indication of the developmental stage may correspond to one of a plurality of developmental stages such an integer between 1 and 46 corresponding to the forty-six (46) HH developmental stages. In another example, the indication of the developmental stage may be a value corresponding to one of a plurality of a stage periods (e.g., stage period including stages HH15-16, stage period including stages HH17-18, stage period including stages HH19-20, and stage period including stages HH21-22). In another example, the indication of the developmental stage may represent a likelihood that, given the blood vessel data, that the egg is in one or more developmental stages or within a stage period (grouping).

A machine learning model may be trained using a training set that includes representations of blood vessel data, and corresponding ground truth data. The training set may include data associated with different eggs and/or different avians. The ground-truth data may be determined using the traditional HH staging method based on recorded microscope images of avian embryos, captured after the opening the egg. For example, after imaging the eggs using an LSCI system described herein (e.g., LSCI system 200 in FIG. 2) and extracting blood vessel data, the eggs are opened up and a commercial wide-field microscope (e.g., MVX10 camera with 1× objective lens sold by Olympus) used to image the embryo body and the blood vessels. One or more experienced biologists determine the stage period and/or developmental stage from the images mainly relying on morphological differences influencing the blood vessel network development of the embryo, such as the heart and the head. As another example, images may be acquired by LED egg candling. Regardless of how ground truth data is obtained, a machine learning model may be trained by providing representations of blood vessel data as input, obtaining an indication of a developmental stage based on the input, and updating weights of the model based on a difference between the developmental stage determined and the ground truth developmental stage.

FIG. 17 is a flowchart of an example process 1700 for training a machine learning model in accordance with some embodiments. Blocks of process 1700 may be executed by one or more computing devices, such as a server device, a desktop computer, a laptop computer, etc. Note that the one or more computing devices may be different than the one or more computing devices that execute blocks of process 1700. In some implementations, blocks of process 1700 may be executed in an order other than what is shown in FIG. 17. In some embodiments, two or more blocks of process 1700 may be executed substantially in parallel. In some embodiments, one or more blocks of process 1700 may be omitted.

Process 1700 can begin at 1702, by obtaining training data, the training data comprising, for a group of avian eggs, representations of blood vessel data, wherein for each training sample, a portion of the obtained data spans an exposure time, and where each training sample includes a corresponding ground truth developmental stage for the egg. As described above, the representations of blood vessel metrics may be a function of time or an average and/or may include extracted blood vessel features associated with the blood vessel metrics. The blood vessel metrics for the training data may be collected using an LSCI system described herein and/or the associated ground truth developmental stage may be determined using a conventional microscope.

At 1704, process 1700 can provide the training data to a machine learning model, where the machine learning model takes, as input, the blood vessel data and generates, as an output, an indication of a developmental stage. Based on the blood vessel data, the machine learning model classifies the egg as in a particular developmental stage or within a stage period. In one example, the indication of the developmental stage may correspond to one of a plurality of developmental stages. In another example, the indication of the developmental stage may be a value corresponding to one of a plurality of a stage periods. In another example, the indication of the developmental stage may represent a likelihood that, given the blood vessel data, that the egg is in one or more developmental stages or within a stage period (grouping).

At 1706, process 1700 can update the machine learning model based on differences between the ground truth developmental stage and the developmental stage determined. For example, weights of the model may be updated based on a loss function that considers the difference between the ground truth developmental stage and the classified developmental stage. Such a machine learning model that accepts blood vessel images may be a DNN or other suitable architecture. Note that model updating may be performed for each training sample, or for a batch of training samples.

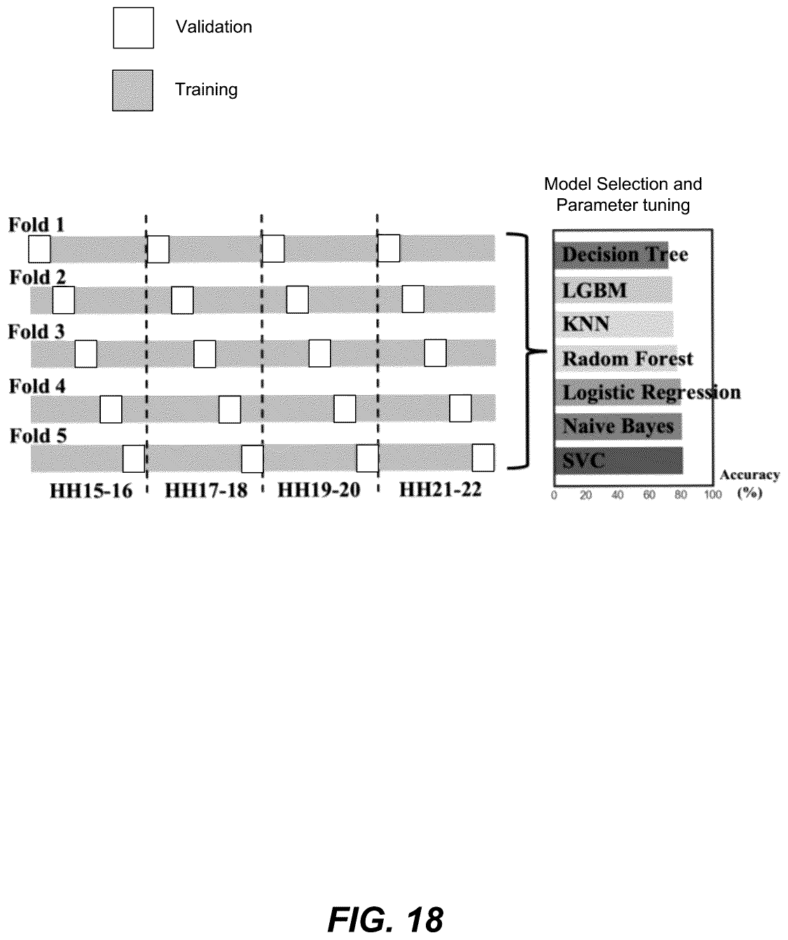

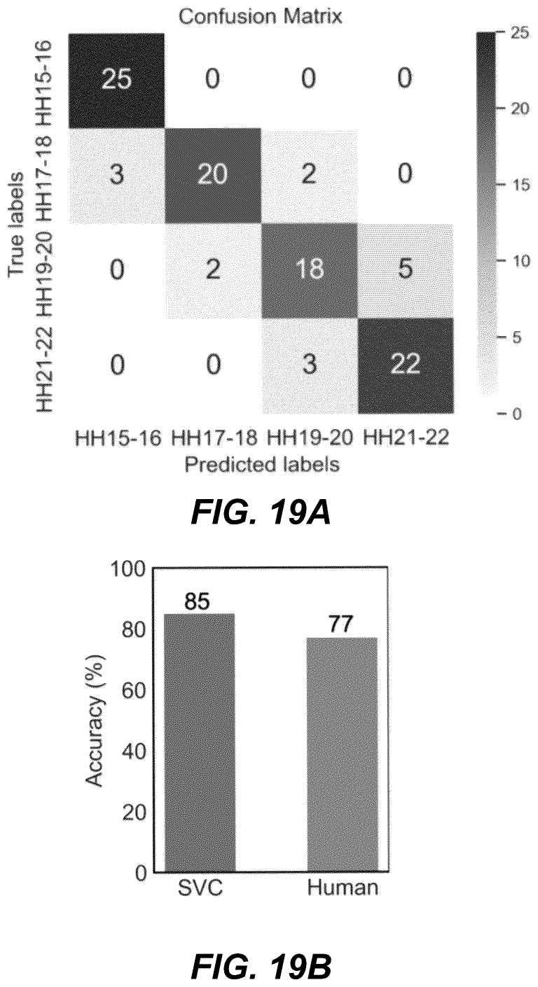

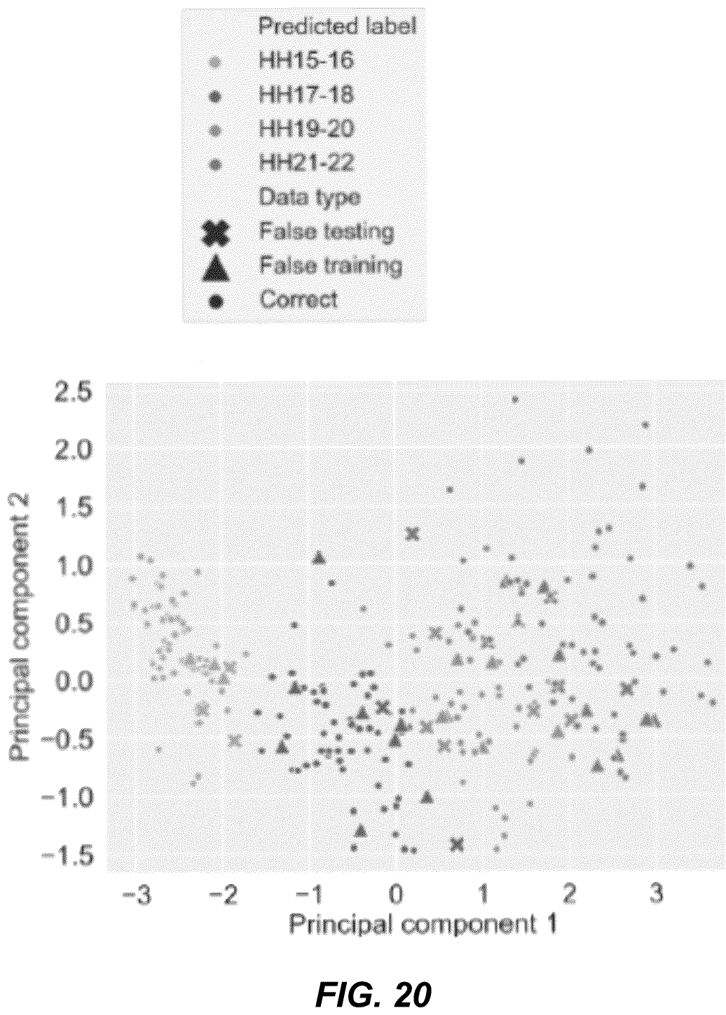

In one example, an LSCI system implements the LSCI process described in connection with FIG. 16 to classify eggs into one of four staged period HH15-16, HH17-18, HH19-20, and HH21-22. The LSCI system also implements the LSCI process 1400 described in connection with FIG. 14 to perform feature extraction. FIG. 11A shows the blood vessel distribution among the four stages. To train the machine learning model to perform classification, a dataset of 260 brown eggs was used with 65 eggs at each developmental stage. The LSCI process 1400 was implemented to extract four features including vessel length, number of branches, vessel area, and standard deviation of BFI as depicted by, for example, the images shown in FIG. 15. The dataset was randomly split into a training set (160 eggs) and a testing set (100 eggs), which were equally balanced among the four stages. The ground-truth stage of each egg was determined by using the traditional HH staging method on recorded microscope images of the chicken embryo, captured after opening the egg. To ensure reliable classification results with the small dataset, five-fold cross-validation was performed on our training set (each fold consists of the combination of 128 eggs for training and 32 eggs for validation) to select the machine learning model and parameters. FIG. 18 is a diagram depicting results of testing Decision Tree, Light Gradient-Boosting Machine (LGBM), K-Nearest Neighbors (KNN), Random Forest, Logistic Regression, Naïve Bayes, and Support Vector Classifier (SVC). As a classifier engine, the results show that the SVC method had higher average accuracy and accuracy consistency across the five folds than the other machine learning models, and had the highest average accuracy of 81.3% and lowest standard deviation of 5% on the validation set. The SVC model was trained using the whole training set and tested the LSCI method using the testing set which was quarantined and unseen by the training process. The results were evaluated using a confusion matrix and average testing accuracy among four stages. FIG. 19A shows a confusion matrix of the results. The confusion matrix shows that the prediction of the developmental stage on a stage period HH15-16 has highest accuracy while the predictions on the three other stages have some misclassifications in the adjacent stages. FIG. 19B shows a plot of the average accuracy of 85% using SVC model in the LSCI method as compared to an average accuracy of 77% from three human tests. FIG. 20 is a plot a 2D visualization of the data point distributions in feature space consisting of the first two principal components using principal component analysis.

### Drug Screening

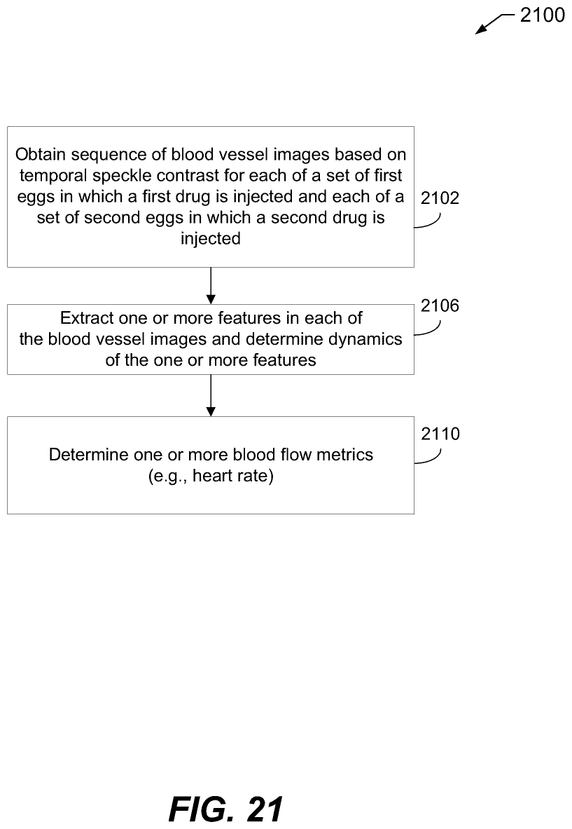

FIG. 21 is a flowchart of an example LSCI process 2100 for drug screening, according to some embodiments. Certain blocks of LSCI process 2100 may be executed by one or more processors of one or more computing devices and other components of an LSCI system such as the LSCI system 4 in FIG. 4. An example of such a computing device is shown in and described below in connection with FIG. 32. In some implementations, blocks of LSCI process 2100 may be executed in an order other than what is shown in FIG. 21. In some embodiments, one or more blocks of LSCI process 2100 may be omitted, and/or two or more blocks may be executed substantially in parallel.

LSCI process 2100 can begin at 2102 by obtaining a sequence of blood vessel images based on temporal speckle contrast for each of a set of first eggs in which a first drug is injected and a set of second eggs in which a second drug is injected. To obtain the sequence of blood vessel images of an egg, an LSCI system may cause, using a laser source (e.g., laser source 410 in FIG. 4), coherent light to be emitted in a first direction onto the egg. Light emitted from the sample is collected from a second direction at an angle from the first direction. The light is collected by an optical system, which propagates collected light to the camera. The LSCI system may obtain, using the camera, a sequence of speckle patterns capturing during an acquisition period. The LSCI system may use the LSCI process 600 shown in and described in connection with FIG. 6 to reconstruct the sequence of blood vessel images of the egg from the sequence of speckle patterns captured using a sliding window across the temporal domain.

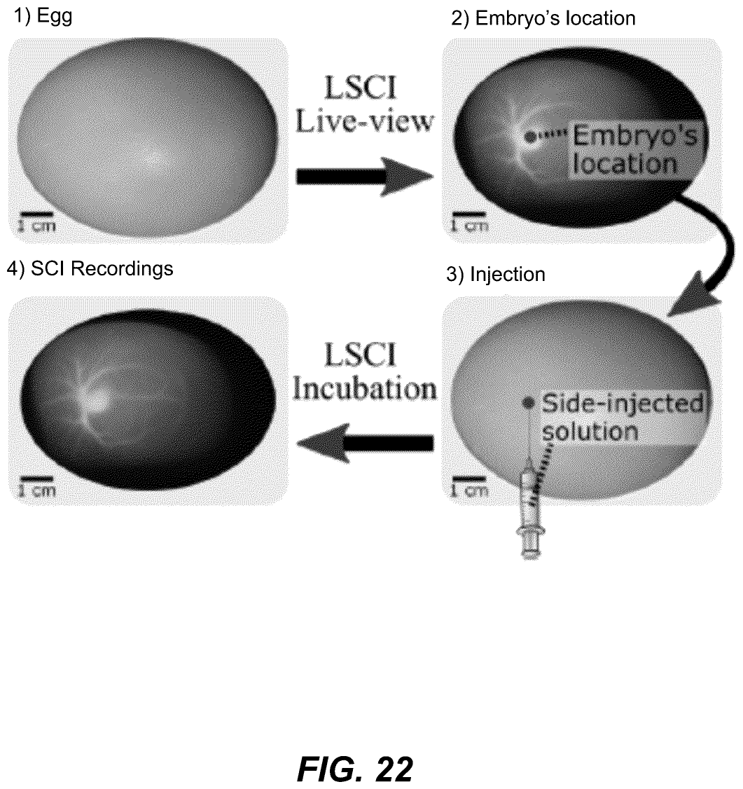

In one example, before imaging, the eggs are injected with a drug by implementing a live view mode to ascertain the position of the embryo's heart and inserted a needle into the eggshell and the drug was injected into the area ventral to the embryo in a volume of 25-150 μL. This may result in a hole in the eggshell having a diameter between 0.6 and 0.8 mm. The hole can be covered by transparent tape to ensure the continued development of the embryo. After injection, the egg may be placed in an incubator (e.g., incubator 440 in FIG. 4) and imaged at regular intervals using a GUI (e.g., GUI 487 in FIG. 4). FIG. 22 is a schematic representation of a procedure for using LSCI imaging to determine the location of avian embryos for injection, according to an embodiment.

At 2106, LSCI process 2100 may extract one or more features from each blood vessel image in the sequence of blood vessel images to evaluate the features at various times post injection. Some examples of blood vessel features that may be extracted include blood vessel length, blood vessel area, number of branches, and/or CAM area. In one example, the LSCI system may implement the LSCI process 1300 or LSCI process 1400 shown in and described in connection with FIGS. 3, and 4 respectively.

At 2106, LSCI process 2100 may extract one or more features from each blood vessel image in the sequence of blood vessel images to determine the dynamics of the features (at various times post injection). Some examples of blood vessel features that may be extracted include blood vessel length, blood vessel area, number of branches, and/or CAM area. In one example, an LSCI system may implement the LSCI process 1300 or LSCI process 1400 shown in and described in connection with FIGS. 3, and 4 respectively to extract the one or more features from each blood vessel image. In one example, a maximum vessel length of all the branches may be calculated as the total length of vessel skeleton excluding the outer ring. As another example, the number of branches may be calculated as the number of branch points plus the number of isolated regions. As another example, the vessel area for all the branches may be calculated as the total number of pixel counts (pixel areas) within the convex hull of all the blood vessels.

At 2108, LSCI process 2100 may determine one or more blood flow metric (e.g., heart rate, blood flow velocity, etc.) from the sequence of blood vessel images. In one implementation, a heart rate may be determined from the sequence of blood vessel images. In this implementation, the LSCI process 2100 determines a heartbeat signal by spatially averaging each blood vessel images using Eqn. 4. The heart rate can be determined by applying a Fourier transform to the heartbeat signal. In other examples, additional or alternative blood vessel metrics may be determined.

### Experimental Data

The LSCI system 400 shown in and described in connection with FIG. 4 was used to test the impact of Nifedipine and Amlodipine (Sigma-Aldrich), two L-Type Ca²⁺ channel antagonists on the chick embryonic heart.

### 1. Nifedipine

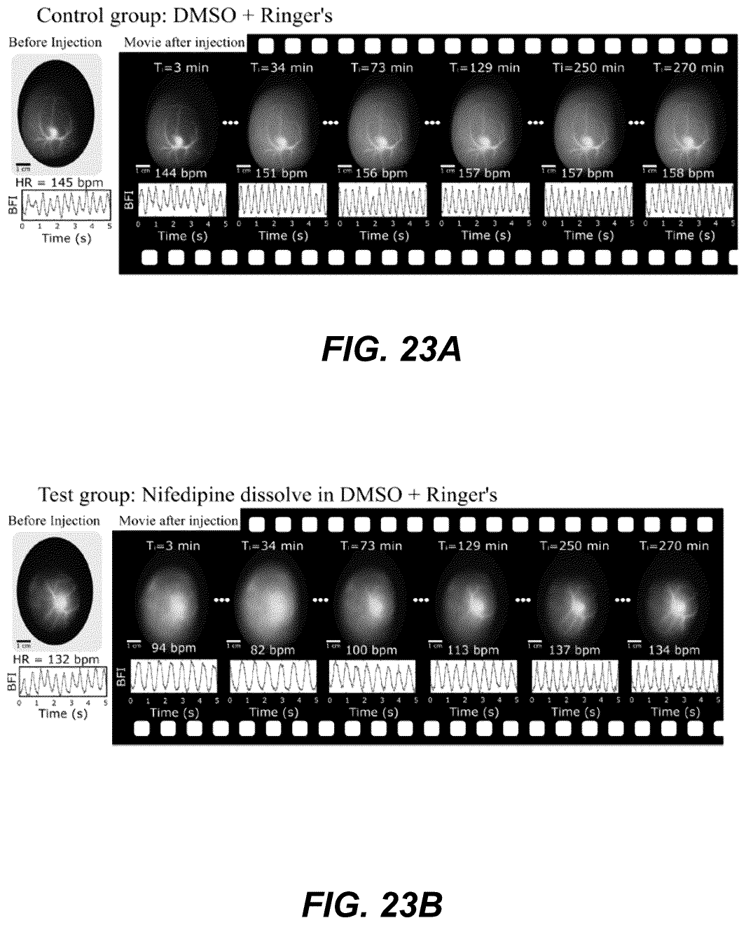

One egg was injected with 50 μL of a solution consisting of 3 nmole of Nifedipine and another egg, corresponding to the control group, was injected with 50 μL of a solution consisting of 1 μL DMSO and 2.5 mL. FIG. 23A depicts images of extraembryonic blood vessels of the control egg generated by the LSCI system 400 in FIG. 4 and images (frames) of a movie showing the dynamics of the blood vessels at time=3 minutes, time=34 minutes, time=73 minutes, time=129 minutes, time=250 minutes, and time=270 minutes. FIG. 23A also includes a time trace of BFI at these times. The time trace of the BFI is indicative of heart rate. As shown in FIG. 23A, there was little change in the heart rate or the pattern of the CAM blood vessels for the control egg over the duration of the recording (270 min). The embryo exhibited almost no change in the heart rate or the pattern of blood vessels over the time of the experiment (250 min).

FIG. 23B depicts images of the extraembryonic blood vessels of an injected with Nifedipine generated by the LSCI system 400 in FIG. 4 before injection and images (frames) of a movie showing the dynamics of the blood vessels after injection at time=3 minutes, time=34 minutes, time=73 minutes, time=129 minutes, time=250 minutes, and time=270 minutes. FIG. 23A also includes a time trace of BFI at these times. The time trace of the BFI is indicative of heart rate. As shown in FIG. 23B, for test egg injected with Nifedipine, the heart rate dropped from 132 beat-per-minute (bpm) to 94 bpm and the CAM's blood vessels were no longer clearly visible due to the decreased blood flow. An hour after injection, the heart rate increased to 100 bpm and by 250 min it had returned to the original pre-injection levels and the blood vessels were again clearly visible.

As shown, the heart rate and vascular structure of the control egg remained consistent across all recordings, while the Nifedipine-injected egg exhibited an initial reduction of blood flow in the CAM which resulted in loss of the vascular image due to undetectable blood flow. This was followed by revival of the heart rate, blood flow, and image of the vascular structure.

### 2. Feature Extraction

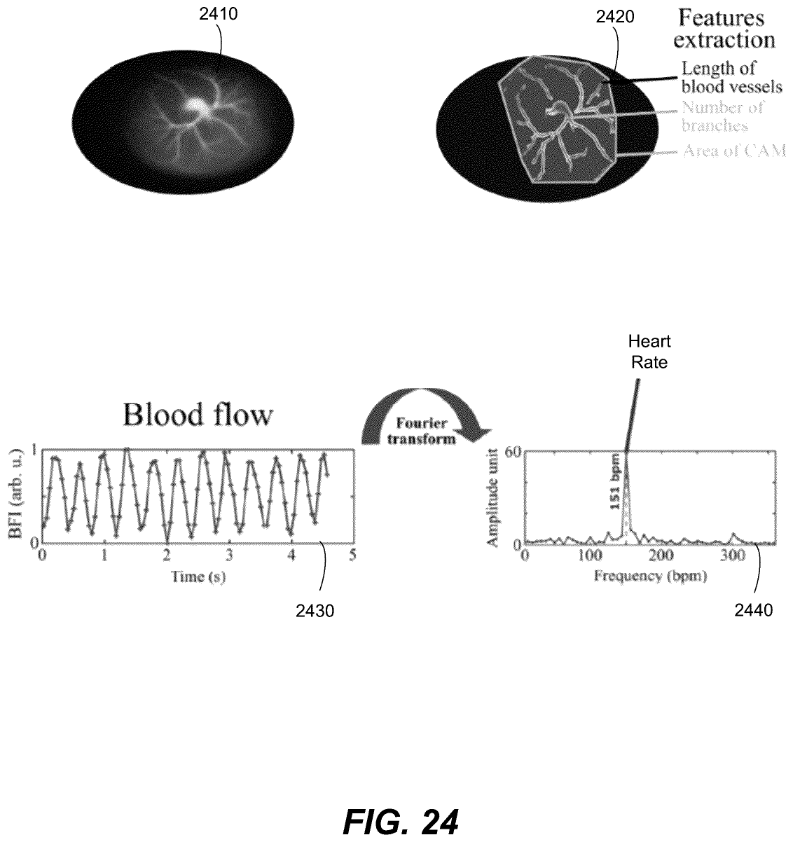

To quantify and characterize the effects of injecting a drug solution onto the CAM, an LSCI system was used to extract four features from the blood vessels images and blood flow graphs. FIG. 24 depicts examples of extraembryonic blood vessel images 2410, 2420 before and after feature extraction, and a heartbeat signal, according to embodiments. Blood vessel skeleton, branches, and the size of the CAM visualized. Extraembryonic blood vessel image 2420 shows the segmenting out of large blood vessels from the images and the identification of the blood vessels skeletons (black lines), the branch points (dots), and CAM area. FIG. 24 also shows a plot 2430 of heartbeat signal of BFI values over time and a plot 2440 of the Fourier transform of the heartbeat signal that shows the heart rate as the peak frequency. The LSCI system implemented the LSCI process 1400 shown in FIG. 14 to extract the features.

In various examples, the length of the blood vessels may be calculated as the total length of the vessel skeleton in the blood vessel image. In various examples, the number of branches may be determined by counting the branch points connecting the blood vessels and isolated vessel segments in the blood vessel images. In various examples, the vessel area may be obtained by calculating the area of the CAM in the blood vessel image. In various examples, the heart rate may be measured from the Fourier transform of the blood flow signal.

### 3. Nifedipine and Amlodipine

Using the LSCI system 400 with an incubator, an LSCI process was used to quantified the heart rate, length of blood vessels, number of branches, and area of the CAM after injecting Nifedipine or Amlodipine into different eggs.

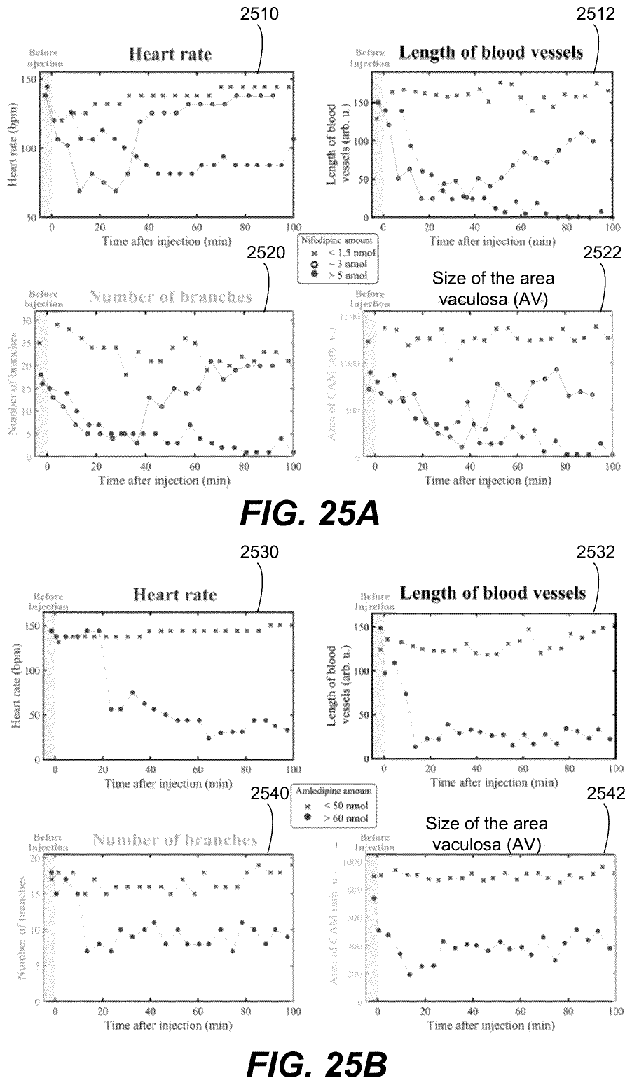

Tests were conducted on five eggs for each of the three Nifedipine concentrations. Tests were also performed on five eggs for each of the two Amlodipine concentrations. FIG. 25A includes a plot of heart rate 2510, a plot of length of blood vessels 2512, a plot of the number of branches 2520, and a plot of the size of the area vaculosa (AV) 2522 of test eggs injected with three different Nifedipine concentrations, according to embodiments. FIG. 25B includes a plot of heart rate 2530, a plot of length of blood vessels 2532, a plot of the number of branches 2540, and a plot of the size of the area vaculosa (AV) 2542 for test eggs injected with two different Amlodipine concentrations, according to embodiments. Although the embryo heart's response to the two drugs was similar, their effective dosage was markedly different.

The chick embryo responded to the Nifedipine in a dose dependent manner as shown in FIG. 25A. At the lowest dose tested (<1.5 nmol), apart from an initial drop during the drug injection in the bpm, the heart rate remained constant throughout the length of the experiment. The embryo developed normally when incubated for 7 days after the experiment. At low doses around 3 nmol, the heart rate dropped over a 40 min period, after which it began to increase reaching normal levels after an hour. At higher doses above 5 nmol, the heart rate of the embryo dropped rapidly reaching its nadir after 20 min. It did not begin to return to normal levels even after 100 min after injection, indicating that the embryo was dying.

Amlodipine was effective on the chick embryo at a dose that was ten times higher than that of Nifedipine. At the lowest dose tested (<50 nmol), apart from an initial drop during the drug injection in the bpm, the heart rate remained constantly low throughout the length of the experiment. The embryo also developed normally when incubated for 7 days after the experiment. At higher doses >60 nmol, the heart rate of the embryo dropped rapidly reaching its nadir after 20 min. At these higher doses, the embryo died at the time of injection, or its later development was heavily compromised.

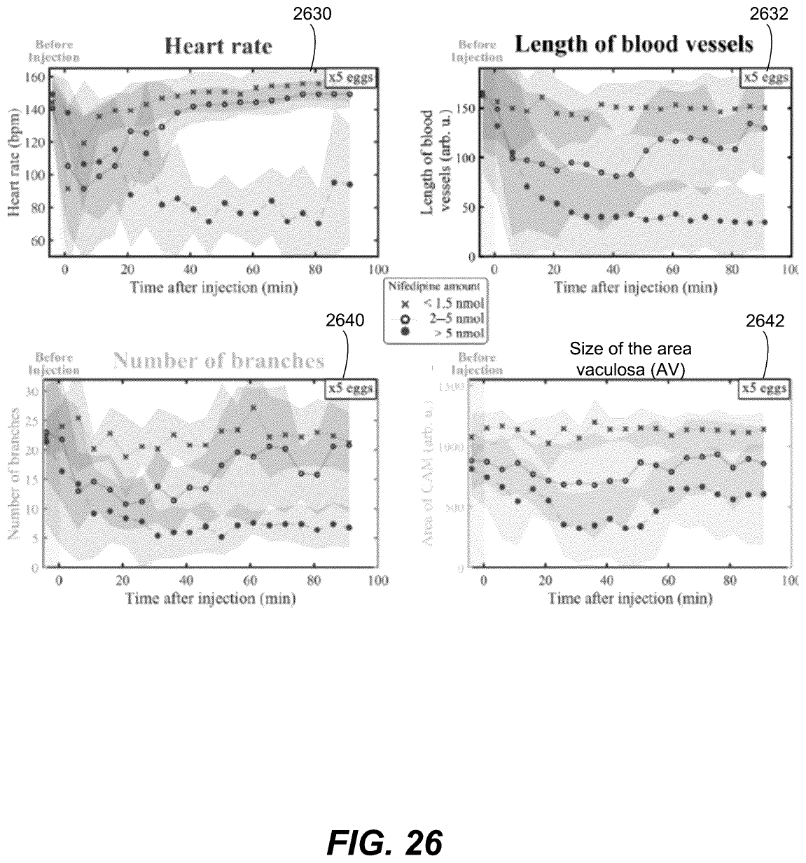

FIG. 26 includes a plot of heart rate 2630, a plot of length of extraembryonic blood vessels 2632, a plot of the number of branches 2640, and a plot of the size of the area vaculosa (AV) 2642 for test eggs injected with different concentrations of Nifedipine, according to embodiments.

### IV. Computational Systems

The nonlinear ultrasound imaging techniques of some implementations described above may be implemented using one or more computing devices. FIG. 32 illustrates an example computing device that may be used, e.g., to implement certain blocks of process 600 of FIG. 6, process 800 of FIG. 8, process 900 of FIG. 9, system 1200 of FIG. 12, process 1300 of FIG. 13, process 1400 of FIG. 14, process 1700 of FIG. 17, and/or process 1800 of FIG. 18, and/or perform functions of LSCI system 200 in FIG. 2, LSCI system 300 in FIG. 3, or LSCI system 400 in FIG. 4.

In FIG. 32, the computing device(s) 3250 includes one or more processors 3260 (e.g., microprocessors), a non-transitory computer readable medium (non-transitory CRM) 3270 in communication with the processor(s) 3260, and one or more displays 3280 also in communication with processor(s) 3260. Processor(s) 3260 is in electronic communication with CRM 3270 (e.g., memory). Processor(s) 3260 is also in electronic communication with display(s) 3280, e.g., to display image data, text, etc. on display 3280. Processor(s) 3260 may retrieve and execute instructions stored on the CRM 3270 to perform one or more functions described above. For example, processor(s) 3260 may execute instructions to perform one or more operations of a nonlinear ultrasound imaging method and/or perform one or more functions of a laser contrast imaging (LSCI) system. The non-transitory CRM (e.g., memory) 3270 can store instructions for performing one or more functions or operations as described above. These instructions may be executable by processor(s) 3260. CRM 3270 can also store raw images, e.g., speckle images, or the like.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, Matlab, or other suitable language/computational software, including low level code, including code written for field programmable gate arrays, for example in VHDL; embedded artificial intelligence computing platform, for example in Jetson. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random-access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

1. A laser speckle contrast imaging method comprising:
(a) causing, using one or more laser sources, light to be emitted into a sample;
(b) obtaining, using one or more light detectors, a sequence of speckle frames indicative of light scattered by one or more dynamic elements within the sample;
(c) applying a temporal sliding window of a plurality of speckle frames to the sequence of speckle frames to determine overlapping sets of speckle frames; and
(d) reconstructing a sequence of dynamic element images based on temporal speckle contrast from corresponding overlapping sets of speckle frames.

2. The method of claim 1, wherein (d) comprises determining a pixel value based on temporal speckle contrast for each pixel in each one of the overlapping sets of speckle frames.

3. The method of claim 2, wherein the pixel value is a blood flow index value calculated from a temporal speckle contrast value determined from the each one of the overlapping set of speckle frames.

4. The method of claim 1, wherein each of the dynamic element images is a blood flow index map.

5. The method of claim 1, wherein the sequence of dynamic element images is viewable as a movie.

6. The method of claim 1, wherein adjacent sets of speckle frames in the overlapping sets of speckle frames are overlapping by at least one speckle frame.

7. The method of claim 1, wherein the plurality of speckle frames in the temporal sliding window comprises at least three speckle frames.

8. The method of claim 1, further comprising subtracting a dark-noise frame from each speckle frames in the sequence of speckle frames.

9. The method of claim 1, further comprising applying a denoising filter to the sequence of dynamic element images.

10. The method of claim 1, further comprising applying a Fourier filter to the sequence of dynamic element images.

11. The method of claim 10, further comprising averaging the dynamic element images to determine a temporally-averaged dynamic element image.

12. (canceled)

13. The method of claim 1,
wherein the sample comprises an avian embryo; and
further comprising:
applying a Fourier filter to the sequence of dynamic element images;
averaging the dynamic element images to determine a temporally-averaged dynamic element image;
extracting one or more blood vessel features from the temporally-averaged dynamic element image; and
predicting a developmental stage of the avian embryo based on the one or more blood vessel features.

14. The method of claim 1,
wherein the sample comprises an avian embryo; and
further comprising:
extracting one or more blood vessel features from the sequence of dynamic element images;
providing the one or more blood vessel features as an input into a trained machine learning model; and
predicting a developmental stage of the avian embryo based on an output of the trained machine learning model.

15. The method of claim 14, wherein the one or more blood vessel features comprise at least one of a blood vessel length, a number of branches, and a blood vessel area.

16. The method of claim 14, wherein the trained machine learning model is a deep neural network (DNN).

17. The method of claim 1, wherein at least one of the one or more laser sources is configured to emit light in a near-infrared wavelength range.

18. A method of drug screening, the method comprising:
(a) injecting a first drug into a set of first eggs;
(b) injecting a second drug into a set of second eggs;
(c) obtaining a sequence of blood vessel images based on temporal speckle contrast of each egg of the first eggs and each of the second eggs;
(d) extracting a plurality of features from each of the blood vessel images for each of the first eggs and second eggs; and
(e) determining a time-varying blood vessel feature metric of each of the features based on the blood vessel images.

19. The method of claim 18, wherein the sequence of blood vessel images of each egg is obtained by:
(i) causing, using one or more laser sources, light to be emitted into each egg;
(ii) obtaining, using one or more light detectors, a sequence of speckle frames indicative of light scattered by one or more blood vessels within each egg;
(iii) applying a temporal sliding window of a plurality of speckle frames to the sequence of speckle frames to determine overlapping sets of speckle frames; and
(iv) reconstructing the sequence of blood vessel images from corresponding overlapping sets of speckle frames.

20-21. (canceled)

22. A laser speckle contrast imaging system for non-invasively imaging a plurality of avian eggs, the laser speckle contrast imaging system comprising:
one or more laser sources;
a plurality of light detectors;
an integrated incubator with a chamber and one or more sample holders within the chamber, each sample holder configured to receive at least one of the avian eggs; and
one or more processors configured to:
(a) cause, using the one or more laser sources, light to be emitted into one of the avian eggs;
(b) obtain, using the one or more light detectors, a sequence of speckle frames indicative of light scattered by one or more dynamic elements within the one avian egg;
(c) applying a temporal sliding window of a plurality of speckle frames to the sequence of speckle frames to determine overlapping sets of speckle frames; and
(d) reconstructing a sequence of blood vessel images of the one avian egg from corresponding overlapping sets of speckle frames.

23. The laser speckle contrast imaging system of claim 22,
wherein the plurality of avian eggs includes a set of first eggs into which a first drug is injected and a set of second eggs into which a second drug is injected;
wherein the one or more processors are further configured to:
perform (a)-(d) on each of the first eggs and each of the second eggs;
extract a plurality of features from each of the sequence of blood vessel images for each of the first eggs and each of the second eggs; and
for each of the first eggs and each of the second eggs, determine a time-varying blood vessel feature metric for each of the extracted features based on the corresponding sequence of blood vessel images.

24. The laser speckle contrast imaging system of claim 23, wherein the extracted features comprise one or more of a blood vessel area, a blood vessel length, a number of branches, and a heart rate.

25. (canceled)

26. The laser speckle contrast imaging system of claim 22, wherein the integrated incubator comprises an egg turning device.

27. (canceled)

28. The laser speckle contrast imaging system of claim 22, wherein (d) comprises determining a pixel value based on temporal speckle contrast for each pixel in each of the overlapping sets of speckle frames.

29. The laser speckle contrast imaging system of claim 28, wherein the pixel value is a blood flow index value calculated from a temporal speckle contrast value determined from a corresponding overlapping set of speckle frames.

30. The laser speckle contrast imaging system of claim 22, wherein the one or more processors are further configured to:
apply a Fourier filter to the sequence of blood vessel images;
average the blood vessel images to determine a temporally-averaged dynamic element image;
extract one or more blood vessel features from the temporally-averaged dynamic element image; and
predict a developmental stage of an avian embryo based on the one or more blood vessel features.

31. The laser speckle contrast imaging system of claim 30, wherein the one or more processors are further configured to:
provide the one or more blood vessel features as an input into a trained machine learning model; and
predict the developmental stage of the avian embryo based on an output of the trained machine learning model.
